SEQUENCE LISTING (31 sequences)

SEQ ID NO: 1 - DNA, 2016 bp, Artificial Sequence
  Description of Artficial Sequence  note = synthetic construct
gaagcgaaatctccccttctacagagtccctccggcgcttcctccaccccgggatacacagaacctcatctcctacggtgctgaagcctgcagcagggcaggatgggcaggagagcagagccgcggagtctgcggcgcgggtgaagagcggcgcgtaattcccgcagcaagattgttccgcgcccgcagcccctggactagcaggatccgaaccccggcggctgcgtgcttataggcgcagacgtcagagagcccgcggcttaaagcgcgtcgcctggctagcgccaccccctagccttcttcaaggcctccagggctgggcccaagcgcccgtcgacggcaccctgggcccagaggactcgcgggcctcatctccaatgattcagaactcacgtccgtcgctgctgcaaccccaagatgtcggagacacggtggaaacgcttatgttacatccggtgatcaaggctttcctgtgtggctccatcagtgggacctgctctaccctccttttccaacctctggatctccttaaaacacgcctacaaaccctccagccctcagatcatgggtctagacgtgttgggatgttggctgtactcttgaaggtggttcgcacggagagtcttttgggcctttggaaagggatgtccccttccattgtgagatgtgtccctggcgttggaatctactttggcactctctactctttgaagcagtatttcttgcgaggccatcccccaaccgccctggagtcagtcatgctgggggtgggctctcgctctgttgcaggggtctgtatgtcacctatcactgtaatcaagacgcgctatgagagtgggaaatatggctatgagagtatctacgctgccctgaggagcatctatcacagtgaggggcaccggggcctcttcagtggcctgacagcaactctccttcgagatgcgcccttctcaggaatctacctgatgttttacaaccagaccaaaaatatagtgcctcatgaccaggtggatgcaacccttattcctattacaaatttcagctgtgggatatttgctggtattctggcctcactggtaactcaacctgcgggtgttatcaaaactcatatgcagctttatccactgaagtttcaatggattggccaagcagtgacacttattttcaaagactatggactacgtggcttcttccaaggtggcatcccccgagccctccgcagaactctaatggcagcaatggcgtggacggtgtatgaagagatgatggccaagatgggcctgaagtcctgaccaagagaggactgggaacgggtgaaatctgttgccctgcctggtttctgccaagggctgctgcttcttactattctgcagtaagatgaagtcctacctggaaaaccaggcagaaattgtgttgcctttgccttcagtaatccccttaaggagaaaatatatggacctgatttcagccttcagaatctccaaaagaggagtcatcaattcatagagcacactagggtgttaggagagagctttgcatactctgagaggctacttggaaaggcattttcccaggagagctctgtcaggtggctgcgcttcagccccacccctacaccacagggtctccttgggtatgttcttgggcaagcaatcacaaagccagagaagctgtaagctgcctgccgggcctgaggagctccaaccagggaagactggatgtgaggagaggagtcactgtcaccaggtcacagactgactgaggtgatggtaggatgaggaggaacagatgcccttctttaattggttctcagttaacttctcagaggctctggagaacgggacagtggctttctagcctctgaatgttccaaataaaattttttggtcttggcccctgtactgttttacctctaaattctggcatttttttttccctgcaattaaagtgctgtactttatttattgaactttatcattgaaagctgtttggaaaacctaaaaaaaaaaaaaaaaa

SEQ ID NO: 2 - protein, 304 aa, Artificial Sequence
  Description of Artficial Sequence  note = synthetic construct
MIQNSRPSLLQPQDVGDTVETLMLHPVIKAFLCGSISGTCSTLLFQPLDLLKTRLQTLQPSDHGSRRVGMLAVLLKVVRTESLLGLWKGMSPSIVRCVPGVGIYFGTLYSLKQYFLRGHPPTALESVMLGVGSRSVAGVCMSPITVIKTRYESGKYGYESIYAALRSIYHSEGHRGLFSGLTATLLRDAPFSGIYLMFYNQTKNIVPHDQVDATLIPITNFSCGIFAGILASLVTQPAGVIKTHMQLYPLKFQWIGQAVTLIFKDYGLRGFFQGGIPRALRRTLMAAMAWTVYEEMMAKMGLKS

SEQ ID NO: 3 - protein, 326 aa, Artificial Sequence
  Description of Artficial Sequence  note = synthetic construct
MGVSAEPRSLSVAGAGLASPVIEKARSALLQSQDVEDTVETLMLHPVIKAFLCGSISGTCSTLLFQPLDLLKTRLQALQPSDLGPRRVGMLAVFLKVVRTESLLGLWKGMSPSIVRCVPGVGIYFGTLYSSKQYFLRGHPPTALESVILGMGSRSVAGVCMSPITVIKTRYESGTYSYESIYAALRSIYCSEGHRGLFRGLTATLLRDAPFSGLYLMFYSQTRTAVLHGTAQLDAALIPLINFSCGIFAGVLASLVTQPADVIKTHMQLSPVKFQWIGQAATLIFKNHGLRGFFHGSVPRALRRTLMAAMAWTVYEEMMARMGLKS

SEQ ID NO: 4 - protein, 326 aa, Artificial Sequence
  Description of Artficial Sequence  note = synthetic construct
MGVSAEPRSLSVARAGLASPVIEKARSALLPSQDVEDTVETLMLHPVIKAFLCGSISGTCSTLLFQPLDLLKTRLQTLQPSDVGPRRVGMLSVFLKVVRTETLLGLWKGMSPSIVRCVPGVGIYFGTLYSSKQYFLRGHPPTALESVILGMGSRSVAGVCMSPITVVKTRYESGAYSYESVYAALRSIYCSEGSRGLFRGLTATLLRDAPFSGLYLMFYSQTRATVLHGADELDAALMPLVNFSCGVFAGILASLVTQPADVIKTHMQLSTVKCQCIGQVATLILKTHGLRGFFHGSVPRALRRTLMAAMAWTVYEEMMAKMGLKS

SEQ ID NO: 5 - protein, 302 aa, Artificial Sequence
  Description of Artficial Sequence  note = synthetic construct
MSNALVVAGDSLVPSNRVSQMHPVFKAFVCGSLSGTCSTLLFQPLDLVKTRIQAHQLSASAAGSRPRMLNLLIKVVRNENILGLWKGVSPSFLRCIPGVGLYFSTLYTLKHHFFSERDPKPLESVMLGAGSRTVAAVCMLPFTVVKTRYESGKYGYNSVYGALKAIYKTEGPRGLFSGLTATLMRDAPFSGIYLMFYTRAKKLAPHDQIDPLFSPVLNFSCGIVAGILASVATQPADVIKTHMQLANEKYHWTGKVALNIYRTQGLTGFFQGGVPRALRRTLMAAMAWTVYEQMMEKMGLKS

SEQ ID NO: 6 - protein, 287 aa, Artificial Sequence
  Description of Artficial Sequence  note = synthetic construct
MEVALAHPALKAFMCGSLSGTCSTLLFQPLDLVKTRLQTLQNNMHPGAPKVGMITVLFNVIRTEKLLGLWKGVSPSFMRCIPGVGIYFSTFYSLKQHYFQEGSPSAGEAVLLGAGARCVAGVAMLPFTVIKTRFESGRYNYISVAGALKSVCQNEGPKALYSGLTATLLRDAPFSGIYVMFYSQAKKALPQEISSSSIAPLVNFGCGVVAGILASLATQPADVIKTHMQVSPALYPKTSDAMRHVYVKHGLSGFFRGAVPRSLRRTLMAAMAWTVYEQLMARMGLKS

SEQ ID NO: 7 - DNA, 24 bp, Artificial Sequence
  Description of Artficial Sequence  note = synthetic construct
gcggaattcggtgttgtcatagcg

SEQ ID NO: 8 - DNA, 24 bp, Artificial Sequence
  Description of Artficial Sequence  note = synthetic construct
cgcggatccctagttctgcatctg

SEQ ID NO: 9 - protein, 15 aa, Artificial Sequence
  Description of Artficial Sequence  note = synthetic construct
GGGGSGGGGSGGGGS

SEQ ID NO: 10 - DNA, 32 bp, Artificial Sequence
  Description of Artficial Sequence  note = synthetic construct
gcgagatctctatgattcagaactcacgtccg

SEQ ID NO: 11 - DNA, 32 bp, Artificial Sequence
  Description of Artficial Sequence  note = synthetic construct
cgcggtacctcaggacttcaggcccatcttgg

SEQ ID NO: 12 - DNA, 26 bp, Artificial Sequence
  Description of Artficial Sequence  note = synthetic construct
gcgggatcctgattcagaactcacgt

SEQ ID NO: 13 - DNA, 26 bp, Artificial Sequence
  Description of Artficial Sequence  note = synthetic construct
cgcctcgagtcaggacttcaggccca

SEQ ID NO: 14 - protein, 16 aa, Artificial Sequence
  Description of Artficial Sequence  note = synthetic construct
RQPKIWFPNRRKPWKK

SEQ ID NO: 15 - protein, 11 aa, Artificial Sequence
  Description of Artficial Sequence  note = synthetic construct
GRKKRRQRPPQ

SEQ ID NO: 16 - protein, 16 aa, Artificial Sequence
  Description of Artficial Sequence  note = synthetic construct
RQIKIWFQNRRMKWKK

SEQ ID NO: 17 - protein, 16 aa, Artificial Sequence
  Description of Artficial Sequence  note = synthetic construct
RQIAIWFQNRRMKWAA

SEQ ID NO: 18 - protein, 9 aa, Artificial Sequence
  Description of Artficial Sequence  note = synthetic construct
RKKRRQRRR

SEQ ID NO: 19 - protein, 21 aa, Artificial Sequence
  Description of Artficial Sequence  note = synthetic construct
TRSSRAGLQFPVGRVHRLLRK

SEQ ID NO: 20 - protein, 26 aa, Artificial Sequence
  Description of Artficial Sequence  note = synthetic construct
GWTLNSAGYLLGKINKALAALAKKIL

SEQ ID NO: 21 - protein, 18 aa, Artificial Sequence
  Description of Artficial Sequence  note = synthetic construct
KLALKLALKALKAALKLA

SEQ ID NO: 22 - protein, 16 aa, Artificial Sequence
  Description of Artficial Sequence  note = synthetic construct
AAVALLPAVLLALLAP

SEQ ID NO: 23 - protein, 10 aa, Artificial Sequence
  Description of Artficial Sequence  note = synthetic construct
VPMLKPMLKE

SEQ ID NO: 24 - protein, 28 aa, Artificial Sequence
  Description of Artficial Sequence  note = synthetic construct
MANLGYWLLALFVTMWTDVGLCKKRPKP

SEQ ID NO: 25 - protein, 18 aa, Artificial Sequence
  Description of Artficial Sequence  note = synthetic construct
LLIILRRRIRKQAHAHSK

SEQ ID NO: 26 - protein, 21 aa, Artificial Sequence
  Description of Artficial Sequence  note = synthetic construct
KETWWETWWTEWSQPKKKRKV

SEQ ID NO: 27 - protein, 18 aa, Artificial Sequence
  Description of Artficial Sequence  note = synthetic construct
RGGRLSYSRRRFSTSTGR

SEQ ID NO: 28 - protein, 15 aa, Artificial Sequence
  Description of Artficial Sequence  note = synthetic construct
SDLWEMMMVSLACQY

SEQ ID NO: 29 - protein, 12 aa, Artificial Sequence
  Description of Artficial Sequence  note = synthetic construct
TSPLNIHNGQKL

SEQ ID NO: 30 - DNA, 26 bp, Artificial Sequence
  Description of Artficial Sequence  note = synthetic construct
gcggaattcggatgcagaacgacgcc

SEQ ID NO: 31 - DNA, 24 bp, Artificial Sequence
  Description of Artficial Sequence  note = synthetic construct
cgcctcgagtcaaaagttctttga

(19) United States
(12) Patent Application Publication
(10) Pub. No.: US 2007/0299011 A1
(43) Pub. Date: Dec. 27, 2007

(54) APPOPTOSIN AND USES THEREOF FOR TREATING NEURODEGENERATIVE DISEASE AND CANCER

(76) Inventors: Yeguang Chen, Beijing (CN); Fangfang Zhou, Beijing (CN); Han Zhang, LaJolla, CA (US); Yunwu Zhang, LaJolla, CA (US); Francesca-Fang Liao, LaJolla, CA (US); Huaxi Xu, LaJolla, CA (US)

Correspondence Address: NEEDLE & ROSENBERG, P.C., SUITE 1000, 999 PEACHTREE STREET, ATLANTA, GA, 30309-3915, US

(21) Appl. No.: 11/751,183

(22) Filed: May 21, 2007

Related U.S. Application Data
(60) Provisional application No. 60/802,036, filed on May 19, 2006.

(51) Int. Cl. A61K 38/00; A61K 48/00; A61P 43/00; C12N 15/00; C12Q 1/02

(52) U.S. Cl. 514/12; 435/29; 435/320.1; 514/44

(57) ABSTRACT

Disclosed herein are compositions and methods relating to the modulation of Appoptosin levels or activity in the treatment of Neurodegenerative disorders or cancer.

### CROSS-REFERENCE TO RELATED APPLICATIONS

This application claims benefit of U.S. Provisional Application No. 60/802,036, filed Mar. 19, 2006, which is hereby incorporated herein by reference in its entirety.

### STATEMENT REGARDING FEDERALLY SPONSORED RESEARCH

This invention was made with government support under Grants NS04673 and AG021173 awarded by the NIH. The government has certain rights in the invention.

### BACKGROUND OF THE INVENTION

Amyloid precursor protein (APP), a key protein in pathogenesis of Alzheimer's Disease (AD), is a type I transmembrane protein which can be cleaved by β- and λ-secretase to release the amyloidogenic β-amyloid (Aβ) peptides and the APP intracellular domain (AID/AICD). While Aβ has been widely believed to initiate pathogenic cascades culminating AD, the physiological functions of AICD remain elusive. Disclosed herein is a pro-apoptotic and anti-amyloidogenic polypeptide identified by its ability to interact with AICD.

### BRIEF SUMMARY OF THE INVENTION

In accordance with the purpose of this invention, as embodied and broadly described herein, this invention relates to the Appoptosin polypeptide and uses thereof in the study and treatment of neurodegenerative disease and cancer.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the sequence identity for 168 (Appoptosin) in human (FLJ20551, SEQ ID NO:2), mouse (SEQ ID NO:3), rat (SEQ ID NO:4), xenopus (SEQ ID NO:5), and zebrafish (SEQ ID NO:6).

FIG. 2 is a diagram of the deletion mutants of 168. The 168(264-304) fragment was pulled out by AICD by yeast-two-hybrid.

FIG. 3 shows 168(264-304) interacts with APP-AICD in yeast-two-hybrid assay. The interaction of AICD-Gal4 activation domain (AD) with 168(264-304) fragment-DNA binding domain (DBD) activates GAL4-driven beta-galactosidase expression in yeast.

FIG. 4 shows Myc-168 and APP were co-transfected into 293T cells and cell lysates subjected to anti-APP (369) IP and anti-myc Western blot (WB) to detect 168-APP interaction (upper panel). Protein expressions are shown in the middle and lower panels.

FIG. 5 shows 168 interacts with APP in IP-WB: APP can interact with multiple regions of 168. Deletion mutants of myc-168 and APP were co-transfected into 293T cells and cell lysates subjected to anti-APP (369) IP and anti-myc WB to detect 168-APP interaction (upper panel). Protein expressions are shown in the middle and lower panels. *118-304AA expression is low and its interaction with APP was still detected here.

FIG. 6 shows 168 interacts with APP-AICD(C99) in cells: APP-AID(C99) interacts with multiple regions of 168. IP-WB the minimal domain of 168 to interact with AICD is 118-264AA. Deletion mutants of HA-168 and myc-tagged APP-AICD(C99) were co-transfected into 293T cells and cell lysates subjected to anti-myc IP and anti-HA WB to detect 168-APP interaction (upper panel). Protein expressions are shown in the middle and lower panels.

FIG. 7 shows Myc tagged 168 and Mt-Ds-Red were cotransfected into HeLa cells and their subcellular localization were detected with anti-myc indirect immunofluorescence and red fluorescence protein with confocal microscopy.

FIG. 8 shows Myc tagged 168 and Ds-Red were cotransfected into HeLa cells and their subcellular localization were detected with anti-myc indirect immunofluorescence and red fluorescence protein.

FIG. 9 shows Myc tagged 168 and Ds-Red were cotransfected into COS7 or HeLa cells and their subcellular localization were detected with anti-myc indirect immunofluorescence and red fluorescence protein.

FIG. 10 shows 293T cells transfected with different amounts of 168 were stained with propidium iodide (PI, DNA staining) and subjected to a FACScan flow cytometer (Becton Dickinson) for cell cycle analysis. sub-G1 indicates apoptotic cells.

FIG. 11 shows 168 expression induces apoptosis in 239T cells: examined by annexin V/PI double staining. Upper right (UR): later apoptotic cells; lower right (LR): early apoptotic cells.

FIG. 12 shows 168 induces cytochrome c release from mitochondria. Mitochrondia and cytosol were isolated from 293T cells transfected with 168 and subjected to immunoblotting with different antibodies: Tubulin serves as a cytosol marker and CoxIV as a mitochondrial marker.

FIG. 13 shows 168 expression results in decrease of the mitochondrial membrane potential, indicating mitochondria dysfunction. MCCP: an ionophore to disrupt proton gradient used as a positive control to decrease mitochondrial membrane potential.

FIG. 14 shows Knockdown of 168 expression with RNAi.

FIG. 15 shows Knockdown of 168 expression with RNAi inhibits Bax-induced apoptosis: examined by annexin V/PI double staining 293T cells were transfected with pro-apoptotic gene Bax with or without 168 siRNA. Upper right (UR): later apoptotic cells; lower right (LR): early apoptotic cells; total apoptotic cells is the sum of UR and LR.

FIG. 16 shows APP/C99/C57 increases 168 protein expression. A. 293T cells transfected with various constructs as indicated were treated with cycloheximide to inhibit protein synthesis. 168 protein levels were detected by anti-myc immunoblotting. Tubulin serves as a loading control. C57 and C99 are APP-AICD forms. B. Quantitation of the data from A.

FIG. 17 shows Northern blot for human 168/FLJ20551. Total RNA Northern blot of human adult normal tissue was purchased BioChain Institute Inc. (Cat # N1234431). Northern blot hybridization was performed following the manufacturer's protocol, using the full-length human 168 sequence as the probe. The results show a band at about 1.8 kb region, corresponding to the putative 168/FLJ20551 mRNA size, from brain and pancreas RNAs but not other tissue samples. In addition, there is a band of 4.4 kb size in brain and pancreas samples, which can be a 168/FLJ20551 spliced variant.

FIG. 18 shows Northern blot for mouse 168/FLJ20551. Total RNA was extracted from different mouse tissues using Trizol reagent (Invitrogen). Equal amounts of RNA were run on formaldehyde gels and then transferred to nylon membranes. Northern blot hybridization was performed following standard protocols, using a 819 bp PCR fragment as the probe. The result show a band of about 2 kb size from variant tissues including brain. In addition, there is a faint band at about 1.5 kb, which can be a spliced variant.

FIG. 19 shows 168/FLJ20551 affects Aβ generation. N2a cells stably expressing human APP695 were transfected with NotchΔE-myc (Notch lacking its ectodomain and carrying a myc tag at its C-terminus) for 1 day. Cells were split equally and transfected with 168 with different dosage for 2 days. Conditional media were collected for sAPPα and secretary Aβ assays. Cells were lysed in 6% SDS and assayed for Notch/NICD conversion and APP metabolism. 168/FLJ20551 and Notch/NICD were detected using myc antibody 9E10. APP AICD was detected using APP C-terminus antibody 369. APP βCTFs, intracellular Aβ, and secretary Aβ in conditional media were immunoprecipitated using antibody 4G8 and detected using antibody 6E10. Soluble APPα were detected using 6E10. α-tubulin was used as control. The results show that overexpression of 168 inhibits Aβ generation in a dose-dependent manner and slightly increases sAPPα. However, overexpression of 168 does not affect the conversion of Notch to NICD (by comparing the ratios of NICD to Notch).

FIG. 20 shows 168/FLJ20551 affects Aβ40 generation. N2a cells stably expressing human APP Swedish mutations were transfected with 168 for 2 days. Secretary Aβ in conditional media were immunoprecipitated using antibody 4G8 and detected using antibody 6E10. The samples were analyzed using Urea gel, which can differentiate variant Aβ species. The results show that overexpression 168 reduces Aβ40 generation in a dose-dependent manner.

FIG. 21 shows tissue expression of 168/FLJ20551. Expression data is from SymAtlas v1.2.4 provided by ©Genomics Institute of the Novartis Research Foundation (GNF).

FIG. 22 shows tissue expression of 168/FLJ20551. Specifically, high levels of 168 were detected in cerebrum, thymus, lung, liver, and spleen. Modest levels were also detected in the cerebellum and heart.

FIG. 23 shows downregulation of 168 inhibits BH3I- and staurosporine-induced caspase 3 cleavage/activation. N2a cells were first subjected to downregulation of 168 by RNAi, and were then treated with apoptotic reagents BH3I and staurosporine. Caspase 3 was cleaved for activation upon apoptotic treatments, but the cleavage of caspase 3 was largely inhibited when 168 was downregulated. In addition, 168 is subjected to cleavage under apoptotic treatments (indicated by*).

FIG. 24 shows APP is at least partially required for 168-mediated apoptosis. APP wild type (WT) and APP/APLP2 double knockout (DKO) mouse fibroblast cells were transfected with control (vector) or 168. Cells were examined by annexin V/PI double staining. 168-induced cell apoptosis in APP/APLP2 DKO cells was dramatically less than that in APP WT cells.

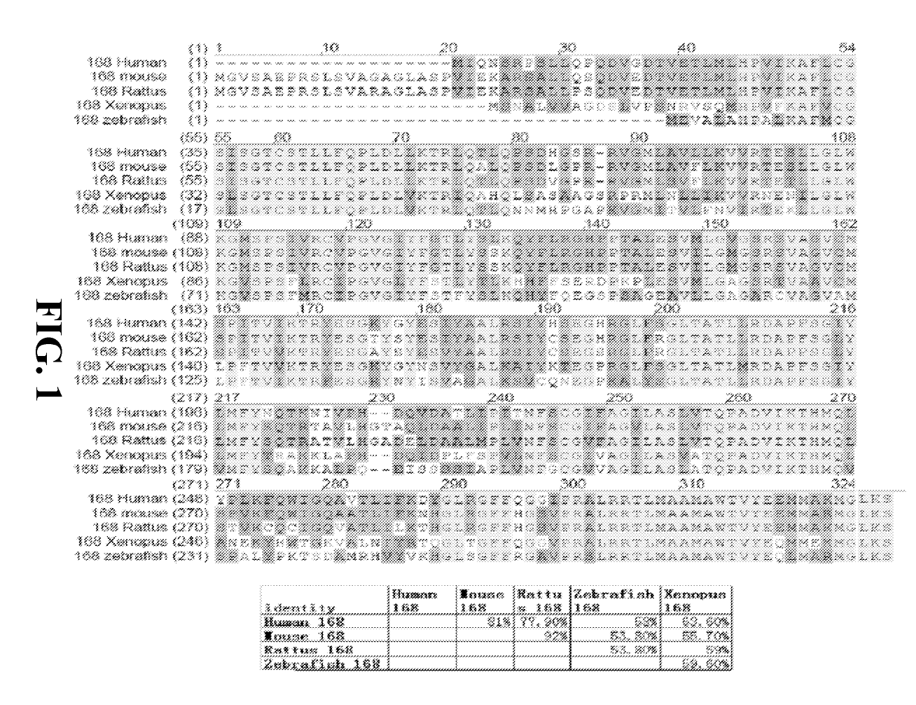

### DETAILED DESCRIPTION OF THE INVENTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a polypeptide” includes a plurality of such polypeptides, reference to “the polypeptide” is a reference to one or more polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### A. APPOPTOSIN

Disclosed herein is a polypeptide referred to herein as “Appoptosin.” Appoptosin is also referred to herein as 168, FLJ20551, YanWang (YW), or by specific accession numbers. For example, an amino acid sequence for human Appoptosin is set forth in Genbank accession number NM_{—}017875 (SEQ ID NO:2). As used herein, “Appoptosin” refers to a nucleic acid encoding Appoptosin. For example, a nucleic acid sequence for human Appoptosin is shown in SEQ ID NO:1.

The YW gene, a putative open reading frame, was identified by the ability of its product to bind amyloid precursor protein (APP), the Alzheimer's Disease (AD)-related gene. However, as disclosed herein, Appoptosin can further induce apoptosis and significantly inhibit the production of β-amyloid peptides. Thus, as disclosed herein, Appoptosin, or fragments or modulators thereof, can be used in the treatment of neurodegenerative disease or cancer. Other uses and variants of the compositions and methods are contemplated and disclosed herein.

### B. METHODS OF TREATING NEURODEGENERATIVE DISORDERS

Provided is a method of treating, preventing, or reducing the risk of developing a neurodegenerative disorder, such as Alzheimer's disease, in a subject comprising administering to the subject a therapeutically effective amount of a modulator of Appoptosin levels or activity. Also provided is a method of treating a subject at risk for a neurodegenerative disorder, such as Alzheimer's disease, comprising administering to the subject a modulator of Appoptosin levels or activity. As used herein, the terms “disorder” and “disease” are used interchangeably to refer to a condition in a subject.

As used herein, the term “Aβ-related disorder” or an “Aβ disorder” is a disease (e.g., Alzheimer's disease) or a condition (e.g., senile dementia) that involves an aberration or dysregulation of Aβ levels. An Aβ-related disorder includes, but is not limited to Alzheimer's disease, Down's syndrome and inclusion body myositis. Thus, the Aβ related disorder can be Alzheimer's disease. The progression of the Aβ related disorder can be slowed or reversed.

Also provided is a method for modulating amyloid-β peptide (Aβ) levels exhibited by a cell or tissue comprising contacting said cell or tissue with an amount of a Appoptosin sufficient to modulate said Aβ levels.

As used herein, a cell or tissue may include, but not be limited to: an excitable cell, e.g., a sensory neuron, motomeuron, or interneuron; a glial cell; a primary culture of cells, e.g., a primary culture of neuronal or glial cells; cell(s) derived from a neuronal or glial cell line; dissociated cell(s); whole cell(s) or intact cell(s); permeabilized cell(s); a broken cell preparation; an isolated and/or purified cell preparation; a cellular extract or purified enzyme preparation; a tissue or organ, e.g., brain, brain structure, brain slice, spinal cord, spinal cord slice, central nervous system, peripheral nervous system, or nerve; tissue slices, and a whole animal. In certain embodiments, the brain structure is cerebral cortex, the hippocampus, or their anatomical and/or functional counterparts in other mammalian species. In certain embodiments, the cell or tissue is an N2a cell, a primary neuronal culture or a hippocampal tissue explant.

Also provided is a method for prevention, treatment, e.g., management, of an Aβ-related disorder, or amelioration of a symptom of an Aβ-related disorder such as Alzheimer's disease. It is understood that the methods described herein in the context of treating and/or ameliorating a symptom can also routinely be utilized as part of a prevention protocol.

Also provided is a method of treating, or ameliorating a symptom of, an Aβ-related disorder comprising administering to a subject in need of such treating or ameliorating an amount of a Appoptosin sufficient to reduce Aβ levels in the subject such that the Aβ-related disorder is treated or a symptom of the AP related disorder is ameliorated.

Neurodegenerative disorders include Alexander disease, Alper's disease, Alzheimer disease, Amyotrophic lateral sclerosis, Ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Huntington disease, Kennedy's disease, Krabbe disease, Lewy body dementia, Machado-Joseph disease, Spinocerebellar ataxia type 3), Multiple sclerosis, Multiple System Atrophy, Parkinson disease, Pelizaeus-Merzbacher Disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, and Tabes dorsalis.

Alzheimer's disease is a progressive neurodegenerative disorder that is characterized by the formation of senile plaques and neurofibrillary tangles containing amyloid β (Aβ) peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

Approximately twenty million people worldwide suffer with dementia that results from Alzheimer's disease. The disease can be early onset affecting individuals as young as 30 years of age, or it can be familial or sporadic. Familial Alzheimer's disease was once thought to be inherited strictly as an autosomal dominant trait; however, this view is changing as more genetic determinants are isolated. For example, some normal allelic variants of apolipoprotein E (ApoE), which is found in senile plaques, can either protect against or increase the risk of developing the disease (Strittmatter et al. (1993) Proc Natl Acad Sci 90:1977-1981).

Amyloid-β (Aβ) peptides are metabolites of the Alzheimer's disease-associated precursor protein, β-amyloid precursor protein (APP), and are believed to be the major pathological determinants of Alzheimer's disease (AD). These peptides consist mainly of 40 to 42 amino acids, Aβ1-40 (“Aβ40”) and Aβ1-42 (“Aβ42”), respectively. Aβ40 and Aβ42 are generated by two enzymatic cleavages occurring close to the C-terminus of APP. The enzymes responsible for the cleavage, β-secretase and γ-secretase, generate the—and C-termini of Aβ, respectively. The amino terminus of Aβ is formed by β-secretase cleavage between methionine residue 596 and aspartate residue 597 of APP (APP 695 isoform numbering) (see, e.g., U.S. Pat. No. 6,440,698; and U.S. Pat. No. 5,744,346).

γ-secretase activity cleaves at varying positions 38-, 40- or 43-residues C-terminal of this β-secretase cleavage to release Aβ peptides (see, e.g., U.S. Patent Application 20020025540). The complete molecular identity of γ-secretase enzyme is still unknown. Presenilin 1, or the closely related presenilin 2, is needed for γ-secretase activity. γ-secretase activity is reduced 80% in cultured cells derived from embryos genetically deleted for presenilin 1. All γ-secretase activity is lost in cells lacking both presenilin 1 and presenilin 2. Peptidomimetic inhibitors of γ-secretase activity can be crosslinked to presenilins 1 and 2, suggesting that these proteins are catalytic subunits for the cleavage. However, γ-secretase activity isolated from cells chromatographs as a large complex>1 M daltons. Recent genetic studies have identified three more proteins required for γ-secretase activity; nicastrin, aph-1 and pen-1. (Francis et al., 2002, Developmental Cell 3(1): 85-97; Steiner et al., 2002, J. Biol. Chemistry: 277(42): 39062-39065; and Li et al., 2002, J. Neurochem. 82(6): 1540-1548). Accumulation of presenilin into high molecular weight complexes is altered in cells lacking these proteins.

A third enzyme, α-secretase, cleaves the precursor protein between the β- and γ-cleavage sites, thus precluding AP production and releasing an approximately 3 kDa peptide known as P3, which is non-pathological. Both β- and α-secretase cleavage also result in soluble, secreted—terminal fragments of APP, known as sAPβ and sAPPα, respectively. The sAPPα fragment has been suggested to be neuroprotective.

In normal individuals, the Aβ peptide is found in two predominant forms, the majority Aβ-40 (also known as Aβ1-40) form and the minority Aβ42 (also known as Aβ1-42) form, each having a distinct COOH-terminus. The major histological lesions of AD are neuritic plaques and neurofibrillary tangles occurring in affected brain regions. Neuritic plaques consist of Aβ peptides, primarily Aβ640 and Aβ42. Although healthy neurons produce at least ten times more Aβ40 compared to Aβ42, plaques contain a larger proportion of the less soluble Aβ42. Patients with the most common form of familial Alzheimer's disease show an increase in the amount of the Aβ42 form. The Aβ40 form is not associated with early deposits of amyloid plaques. In contrast, the Aβ42 form accumulates early and predominantly in the parenchymal plaques and there is strong evidence that Aβ42 plays a major role in amyloid plaque deposits in familial Alzheimer's disease patients (Roher et al., 1993, Proc. Natl. Acad. Sci. USA 90:10836; Iwatasubo, T., et al., 1994 Neuron 13:45; Yamaguchi et al, 1995, Amyloid Int. J. Clin. Invest. 2:7-16; and Mann et al., 1996 Am. J. Pathol. 148:1257).

Mutations in four genes are known to predispose an individual to Alzheimer's disease: ApoE, amyloid precursor protein (APP), presenilin-1, and presenilin-2 (Selkoe (1999) Nature 399:A23-A31). The e4 allele of the ApoE gene confers increased risk for late onset Alzheimer's disease. β-amyloid protein (Aβ) is the major component of senile plaques, and it is normally formed when β- and γ-secretase cleave APP. In Alzheimer's disease patients, large quantities of Aβ are generated and accumulate extracellularly in these neuropathological plaques. Efforts to understand the mechanism underlying Aβ deposition have recently focused on the APP-cleaving secretase. In fact, two yeast aspartyl proteases have been shown to process APP in vitro (Zhang et al. (1997) Biochem Biophys Acta 1359:110-122). Evidence using peptidomimetic probes further confirms that the secretase is an intramembrane-cleaving aspartyl protease (Wolfe et al. (1999) Biochemistry 38:4720-4727). The presenilin-1 gene is a candidate for the γ-secretase that cleaves the APP carboxyl terminus. Several lines of evidence support the involvement of presenilins in the disease process. Presenilin can be coimmunoprecipitated with APP, and mutations in the presenilin genes increase production of the 42-amino acid peptide form of Aβ. These missense point mutations result in a particularly aggressive, early onset form of the disease (Haaas and DeStrooper (1999) Science 286:916-919).

The proteases, BACE1 and BACE2 (β-site APP cleaving enzymes 1 and 2) which appear to be β-secretase, are potential therapeutic targets because of their ability to cleave APP. Vassar et al. (1999; Science 286:735-741) have found that BACE1 is an aspartyl protease with β-secretase activity which cleaves APP to produce Aβ peptide in vitro. It is expressed at moderate levels across all brain regions and is concentrated in neurons but not in glia. BACE2, which has 52% amino acid identity with BACE1, has been described by Saunders et al. (1999; Science 286:1255a). Whereas BACE1 maps to the long arm of chromosome 11, BACE2 maps to the Down syndrome region of chromosome 21 (Acquati et al. (2000) 468: 59-64; Saunders et al. supra). This location is significant because middle-aged Down syndrome patients have enhanced β-amyloid deposits. Other members of the BACE family can also participate in this APP cleavage: the amino terminals of Aβ peptides appear to be cleaved heterogeneously indicating that there can be several β-secretase involved in APP processing (Vassar (1999) Science 286:735-741).

Associations between Alzheimer's disease and many other genes and proteins have been reported. Fetal Alzheimer antigen (FALZ) and synuclein a (SNCA) are found in brain plaques and tangles. Inheritance of some gene polymorphisms is also linked to increased risk of developing the disease. For example, a polymorphism in the gene encoding β2-macroglobulin, a protein that can act as a protease inhibitor, is associated with increased risk for developing a late-onset form of Alzheimer's disease.

One hundred years ago Alois Alzheimer described the major behavioral and neuropathological features of the neurodegenerative disorder bearing his name. AD is characterized clinically/behaviorally by progressive impairment of memory and cognition. Neuropathological and neurobiological changes associated with this slow progression of clinical symptoms include accumulation of amyloid plaques and neurofibrillary tangles (NFTs) (Gearing M. et al., The Consortium to Establish a Registry for Alzheimer's Disease (CERAD). Part X. Neuropathology confirmation of the clinical diagnosis of Alzheimer's disease. Neurology. 1995;45(3 Pt 1):461-466) gliosis (Unger J W., Microscopy Res. Technique. 1998; 43:24-28), reduced dendritic plasticity relative to normal aged (Buell S J. Coleman P D., Science. 1979; 206(4420):854-856), Flood D G. et al., Brain Research. 1985; 345(2):366-368, Flood D G., et al., Brain Research. 1987; 402(2):205-216), and reduced density of neurons (Coleman P D. Flood D G., et al., Neurobiology of Aging. 1987; 8(6):521-545), Terry R D, et al., 1987; 21:530-539, West M J, et al., Lancet. 1994; 344:769-772) and synapses (Scheff S W. et al., Neurobiology of Aging. 1990;11(1):29-37).

Studies of altered gene expression in Alzheimer's disease brain tissue have shown a general reduction of message level estimated at about 35% (Doebler J A, et al., Journal of Neuropathology & Experimental Neurology. 1987; 46(1):28-39), (Griffin W S, et al., Alzheimer Disease & Associated Disorders. 1990; 4(2):69-78), (Harrison P J, et al., Psychological Medicine. 1991;21:855-866). Against this background of a general reduction of mRNA, selected studies have demonstrated increased as well as decreased expression of a wide variety of genes. Some gene classes affected in Alzheimer's disease are expressed in a neuron specific manner. These especially include decreased expression of selected genes that are related to synaptic structure and function and the neuronal cytoskeleton (Ginsberg S D. et al., Annals of Neurology. 2000; 48(1):77-87), (Yao P, et al., Journal of Neuroscience. 1998; 18(7):2399-2411). Other classes of genes whose expression is altered in AD include those related to the cell cycle (Arendt T., Neurobiology of Aging. 2000; 21(6):783-796), (Husseman J W., et al., Neurobiology of Aging. 2000; 21(6):815-828), (Nagy Z., et al., Neurobiology of Aging. 2000; 21(6):761-769), (Vincent I, et al., J. Neurosci. 1997; 17:3588-3598) and inflammatory/stress responses (for a review, see (Akiyama H., et al., Neurobiology of Aging. 2000; 21(3):383-421)). These gene classes are expressed in a variety of cell types that reside outside the nervous system including leukocytes (Wakutani Y. et al., Dementia. 1995; 6(6):301-305), monocytes (Jung S S. et al., Neurobiology of Aging. 1999; 20(3):249-257), and epithelial cells (Schmitz A., et al., Histochemistry & Cell Biology. 2002;117(2):171-180) as well as other cell types.

Multivariate analysis of profiles of expression of multiple gene products (messages) by single neurons or homogenates from postmortem human brain can be used to distinguish Alzheimer's disease from control samples (Cheetham J E., et al., Journal of Neuroscience Methods. 1997;77(1):43-48, Chow, N., et al., Proc. Natl. Acad. Sci. USA. 1998;95:9620-9625).

Symptoms of Aβ-related disorders are well known to those of skill in the art. For example, symptoms of Alzheimer's disease are well known in the art and can include, e.g., memory loss, mild cognitive impairment, cognitive decline, severe cognitive impairment and personality changes that result in loss of functional ability, e.g., over the course of a decade. In debilitated states, patients usually exhibit severe impairment, and retain only vegetative neurologic function. Symptoms of Alzheimer's disease can also include certain art-known neuropathological lesions, including intracellular neurofibrillary tangles and extracellular parenchymal and cerebrovascular amyloid.

### C. METHODS OF INDUCING APOPTOSIS

Provided is a method of inducing apoptosis in a target cell comprising contacting the target cell with a therapeutic amount of a modulator of Appoptosin levels or activity. The ability of an agonist to induce apoptosis can be confirmed by culturing cells such as the human leukemia cell line Jurkat (American Type Culture No. TIB-152) and astrocytoma cell line 1321N1 in medium in which the test sample has been added. The survival rate can be determined by, for example, using an ATPLite assay.

Programmed cell death (PCD) is an important mechanism in both development and homeostasis in adult tissues for the removal of either superfluous, infected, transformed or damaged cells by activation of an intrinsic suicide program. One form of PCD is apoptosis, which is characterized by maintenance of intact cell membranes during the suicide process so as to allow adjacent cells to engulf the dying cell so that it does not release its contents and trigger a local inflammatory reaction. Cells undergoing apoptosis usually exhibit a characteristic morphology, including fragmentation of the cell into membrane-bound apoptotic bodies, nuclear and cytoplasmic condensation and endolytic cleavage of the DNA into small oligonucleosomal fragments (Steller, 1995). The cells or fragments are then phagocytosed by macrophages.

The mitochondrion (intrinsic) apoptotic pathway initiates with signaling from pro-apoptotic proteins from the Bcl-2 family such as Bax, which trigger the release of cytochrome c in the induction phase. Cytochrome c becomes a key regulator in the effector phase because once it is released from the mitochondria the cell is irreversibly committed to death. Cytochrome c then associates with Apaf-1, caspase-9, and ATP to form an apoptosome. The apoptosome then activates caspase-3, which in turns activates the caspase cascade and the degradation phase of apoptosis (Harrison & Link 2003). Overall, the Bcl-2 family proteins in this pathway are of major importance for study as they regulate apoptosis, where anti-apoptotic members such as Bcl-2 and Bcl-X inhibit and pro-apoptotic members such as Bax and Bad induces cytochrome c release (Fadeel 2003).

The death receptor (Extrinsic) apoptotic pathway is initiated by binding of death activators (i.e. FasL, TNF) to their respective transmembrane death receptors. This binding allows interaction with the cytoplasmic adaptor protein FADD, and procaspase-8 that activates membrane-proximal activator caspases 8. The activation of caspase 8, similar to caspase 9 in intrinsic pathway leads to effector caspases activation and the degradation phase of apoptosis (Ethell & Buhler, 2003). Apoptosis-Inducing Factor (AIF) Pathway Under inducing signal, Apoptosis-inducing factor (AIF) located in the intermembrane space of the mitochondria is released and migrates into the nucleus. Once inside the nucleus it binds to DNA and triggers the destruction of DNA and the degradation phase of apoptosis.

The cause of cancer cell development is damage to DNA. Under normal circumstances, when DNA gets damaged, either the cell dies by apoptosis or the DNA is able to repair itself. In cancer cells, the damaged DNA is not repaired and the apoptotic pathways are disturbed.

The methods and compositions provided herein can be used in the treatment of diseases associated with inappropriate survival or proliferation of cells, including those attributable to dysregulation of the apoptosis systems in cancer or in inflammatory and autoimmune diseases. Thus, provided is a method of treating a subject with cancer or with an autoimmune or inflammatory disease, comprising administering to the subject a therapeutic amount of a modulator of Appoptosin levels or activity.

Some cancers arise from the dysfunction in the apoptotic pathway. In the intrinsic pathway, a very common cause of tumorgenesis is mutation of the p53 protein. Besides regulating apoptosis, p53 also regulates the check points in the cell cycle, DNA repair, senescence and genomic integrity. Any mutation that causes p53 to lose any of its function will induce tumorgenesis by letting the cell grow indefinitely without any regulation. Another important factor in tumorgenesis is the balance between the proapoptotic and antiapoptotic members of Bcl-2 family. In a tumor cell, a mutation of Bcl-2 gene that results in increased expression will suppress the normal function of the proapoptotic proteins, BAX and BAK. On the other hand, if a mutation on the BAX or BAK genes cause a downregulation of expression then the cell will also lose its ability to regulate apoptosis, again causing tumorgenesis. Thus, in some aspects, cancers that can be treated by the disclosed methods are those involving dysregulated intrinsic apoptosis.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

In some aspects, the target cell of the disclosed method is a blood cell. Thus, provided herein is a method of treating a leukemia comprising administering to a blood cell a modulator of Appoptosin levels or activity. In some aspects, the blood cell can be a bone marrow cell. In some aspects, the bone marrow cell can be an early erythroid cell. Thus, in some aspects, the blood cell can be a CD71+ cell. In some aspects, the bone marrow cell can be an hemangioblast. Thus, in some aspects, the bone marrow cell can be a CD105+ cell. In some aspects, the blood cell can be a hematopoietic stem cell. In some aspects, the blood cell can be a T-cell. Thus, in some aspects, the blood cell can be a CD4+ cell. Thus, in some aspects, the blood cell can be a CD8+ cell. In some aspects, the blood cell can be a B-cell. Thus, in some aspects, the blood cell can be a CD 19+ cell. In some aspects, the blood cell can be an NK cell. Thus, in some aspects, the blood cell can be a CD56+ cell. In some aspects, the blood cell can be a lymphoblast.

Compounds disclosed herein may also be used for the treatment of precancer conditions such as cervical and anal dysplasias, other dysplasias, severe dysplasias, hyperplasias, atypical hyperplasias, and neoplasias.

Inflammatory and autoimmune diseases illustratively include systemic lupus erythematosus, Hashimoto's disease, rheumatoid arthritis, graft-versus-host disease, Sjögren's syndrome, pernicious anemia, Addison disease, scleroderma, Goodpasture's syndrome, Crohn's disease, autoimmune hemolytic anemia, sterility, myasthenia gravis, multiple sclerosis, Basedow's disease, thrombopenia purpura, insulin-dependent diabetes mellitus, allergy, asthma, atopic disease, arteriosclerosis, myocarditis, cardiomyopathy, glomerular nephritis, hypoplastic anemia, rejection after organ transplantation. Cancers include numerous malignancies of lung, prostate, liver, ovary, colon, cervix, lymphatic and breast tissues. Thus, the provided compositions and methods can further be used to target and selectively induce apoptosis in activated immune cells including activated lymphocytes, lymphoid cells, myeloid cells, and rheumatoid synovial cells (including inflammatory synoviocytes, macrophage-like synoviocytes, fibroblast-like synoviocytes) and in virally infected cells (including those infected with HIV, for example).

As used herein, “subject” includes, but is not limited to, a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the terms “treat”, “treatment” and “treating” refer to the amelioration of a Aβ-related disorder that results from the administration of one or more compounds that lower Aβ levels exhibited by a cell or tissue. It also refers to the managing of the disorder, or the slowing and/or reversing of the progression of the disorder.

By “treat” or “treatment” is meant a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for treating Alzheimer's disease can result in a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. As another example, a disclosed method for treating Alzheimer's disease can result in the amelioration of a Aβ-related disorder that results from the administration of one or more compounds that lower Aβ levels exhibited by a cell or tissue. Treat and treatment can also refer to the managing of the disorder, or the slowing and/or reversing of the progression of the disorder.

As used herein, modulating the levels of Appoptosin includes both methods of modulating intrinsic or extrinsic expression of Appoptosin as well as to methods of administering exogenous Appoptosin.

As used herein, “activities” of a protein include, for example, transcription, translation, intracellular translocation, secretion, phosphorylation by kinases, cleavage by proteases, homophilic and heterophilic binding to other proteins, and degradation.

Also contemplated are modulators that target other molecules either upstream or downstream of Appoptosin in the relevant molecular pathways. For example, the modulator of the disclosed method can target Appoptosin binding sites on APP/AICD. Similarly, the modulator can target the trafficking, stability (degradation), or conformational changes resulted from Appoptosin activity. The modulator can also target Appoptosin-mediated molecules involved in apoptosis, such as, for example, cytochrome C, bcl, Bax, CoxIV, nuclear receptors, or caspases.

“Modulate” or “modulating” refers to an increase or decrease in an activity. This can include but is not limited to the inhibition or promotion of an activity, condition, disease, or response or other biological parameter.

As used herein, the terms “therapeutic amount” and “pharmaceutically effective amount” are synonymous. One of skill in the art could readily determine the proper therapeutic amount.

In the treatment of disease, e.g., cancer, autoimmune and inflammatory diseases, combinations of treatment can also be used. For example, radiotherapy can be combined with or without other therapeutic agents. One skilled in the art would adapt the form of radiotherapy to the disease.

As another example, the modulator of the provided methods and compositions can be administered in conjunction with other therapeutic agents. As used herein a “therapeutic agent” is a compound or composition effective in ameliorating a pathological condition.

Examples of therapeutic agents include chemotherapeutic agents, pro-apoptotic agents, anti-inflammatory agents, Disease Modifying Anti Rheumatic Drug (DMARDs), antibodies, members of TNF family, antiviral agents, anti-opportunistic agents, antibiotics, immunosuppressives, immunoglobulins, anti-malarial agents, anti-rheumatoid arthritis agents, cytokines, chemokines, growth factors, and anti-cancer compounds. An anti-cancer compound is a compound or composition effective in inhibiting or arresting the growth of an abnormally growing cell. Illustrative examples of anti-cancer compounds include: bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine and vinblastine. Further examples of anti-cancer compounds and therapeutic agents are found in The Merck Manual of Diagnosis and Therapy, 15th Ed., Berkow et al., eds., 1987, Rahway, N. J. and Sladek et al. Metabolism and Action of Anti-Cancer Drugs, 1987, Powis et al. eds., Taylor and Francis, New York, N.Y.

### D. MODULATORS OF APPOPTOSIN

The modulators of the provided methods can affect the levels of Appoptosin by exogenous administration of Appoptosin or by modulating intrinsic or extrinsic expression of Appoptosin. Other variations and combinations of these methods are contemplated and disclosed herein.

1. Peptides

The provided methods can comprise administering a polypeptide comprising Appoptosin, or a fragment or conservative variant thereof.

The disclosed polypeptide can comprise an amino acid sequence having, for example, at least about 70, 75, 80, 85, 90, 95%, or 100% sequence identity to the amino acid sequence set forth in SEQ ID NO:2. The disclosed polypeptide can comprise an amino acid sequence having, for example, at least about 70, 75, 80, 85, 90, 95%, or 100% sequence identity to the amino acid sequence set forth in SEQ ID NO:3. The disclosed polypeptide can comprise an amino acid sequence having, for example, at least about 70, 75, 80, 85, 90, 95%, or 100% sequence identity to the amino acid sequence set forth in SEQ ID NO:4. The disclosed polypeptide can comprise an amino acid sequence having, for example, at least about 70, 75, 80, 85, 90, 95%, or 100% sequence identity to the amino acid sequence set forth in SEQ ID NO:5. The disclosed polypeptide can comprise an amino acid sequence having, for example, at least about 70, 75, 80, 85, 90, 95%, or 100% sequence identity to the amino acid sequence set forth in SEQ ID NO:6.

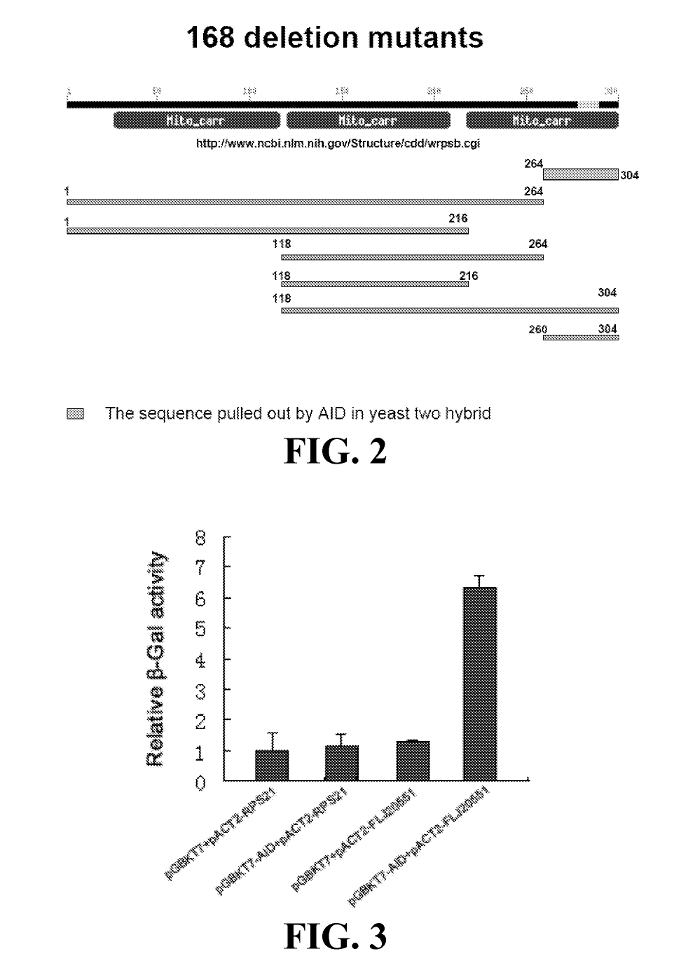

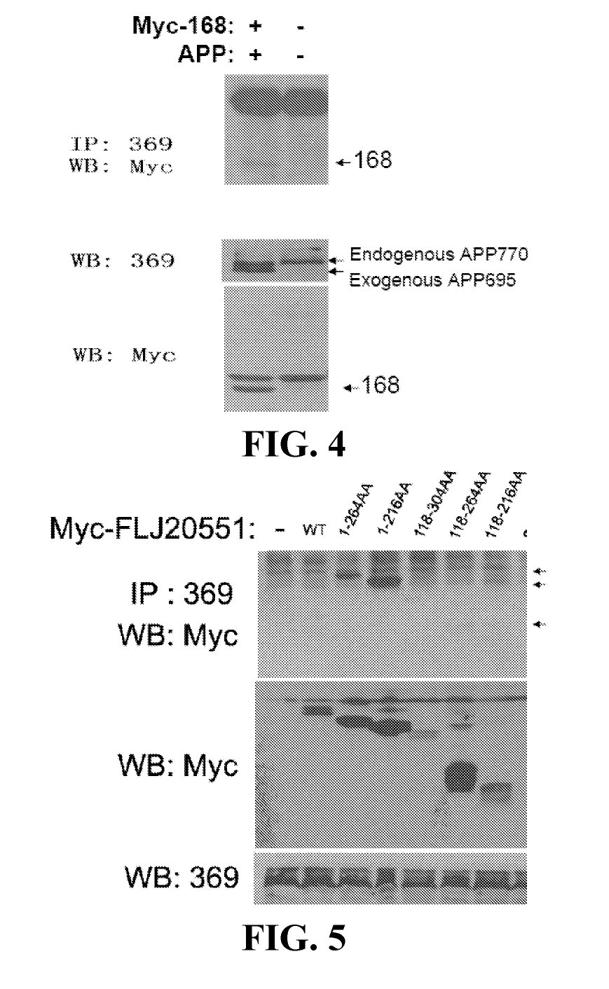

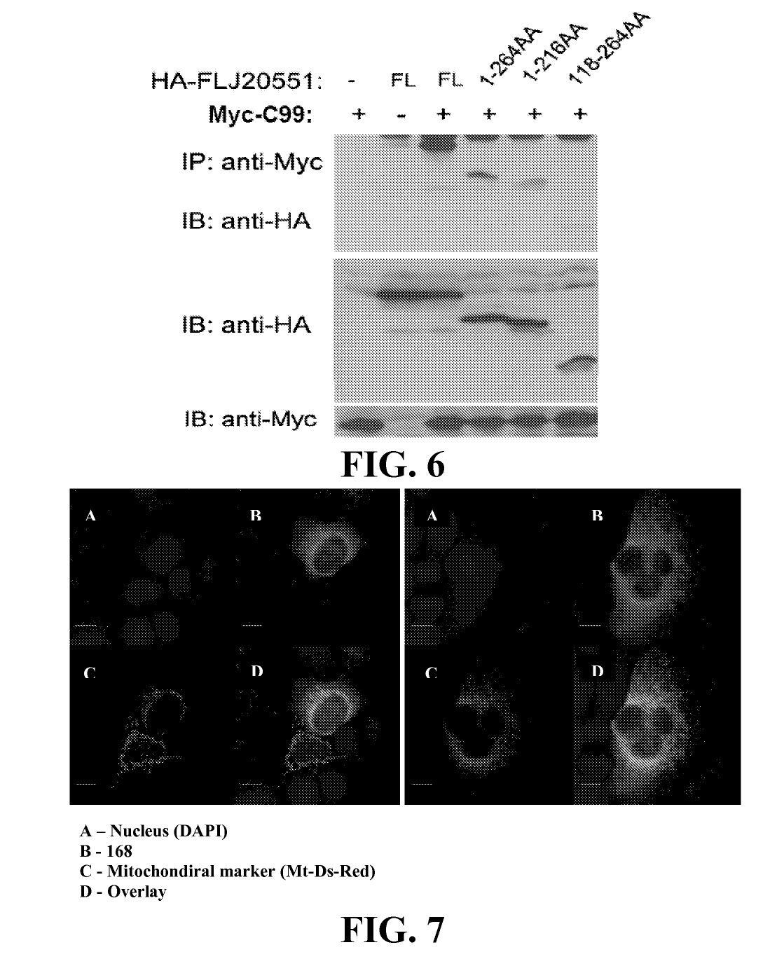

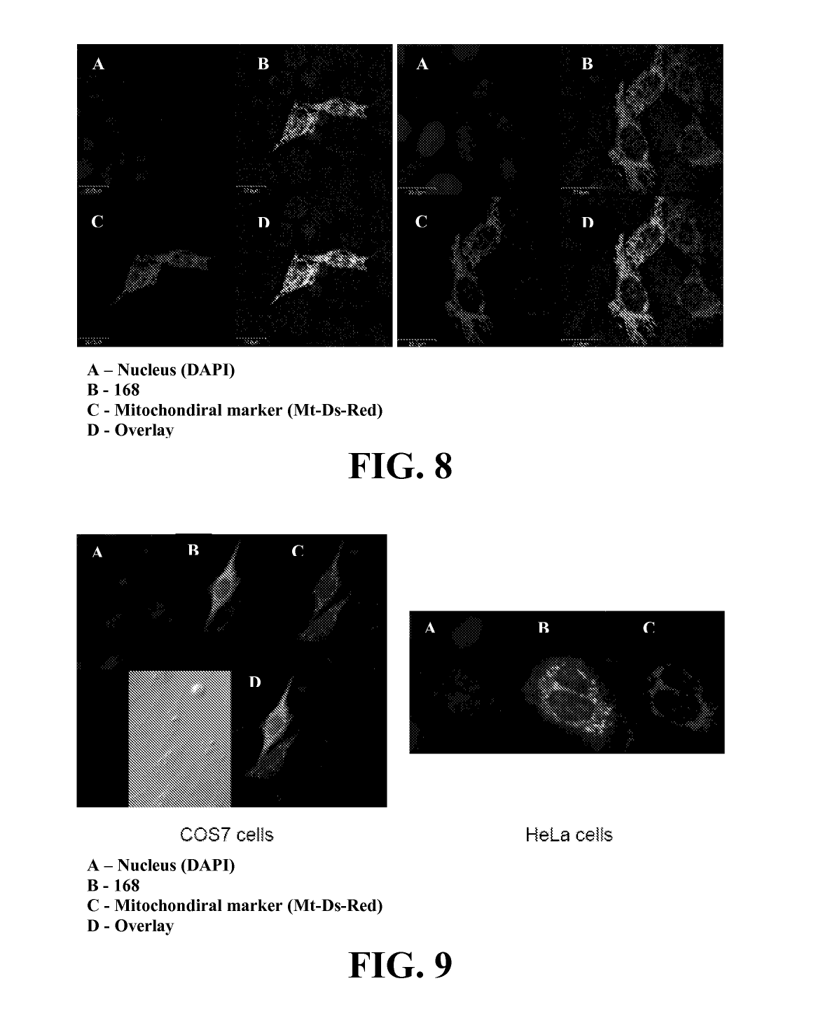

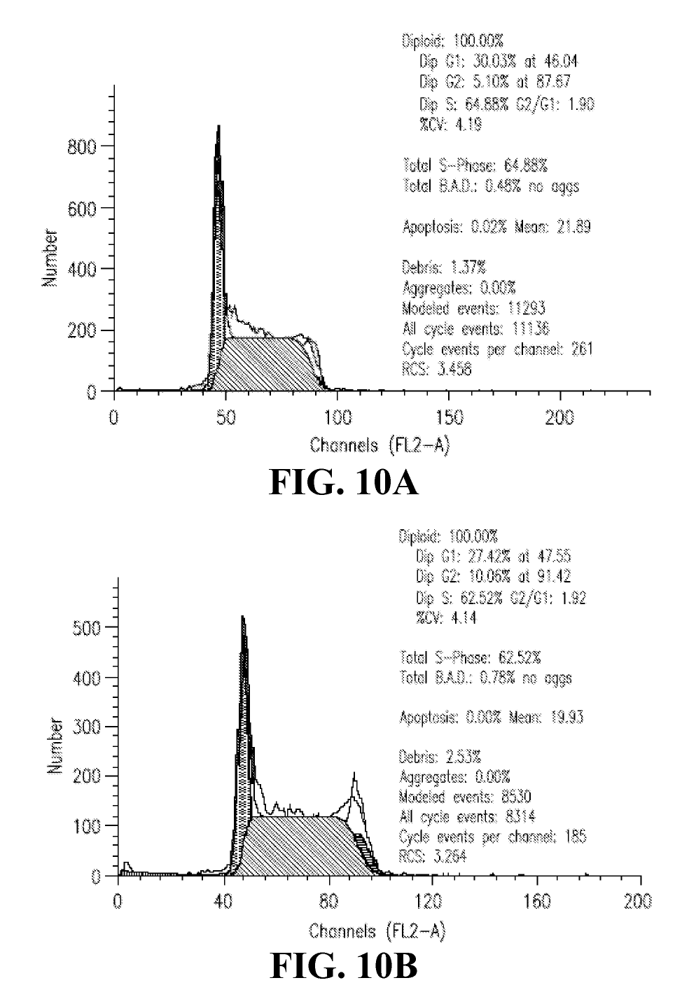

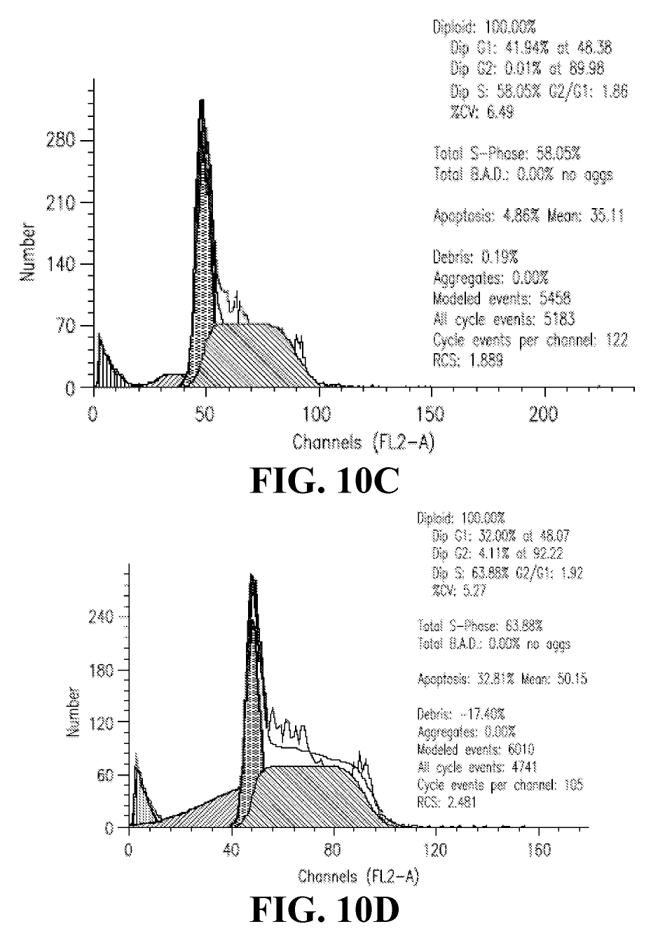

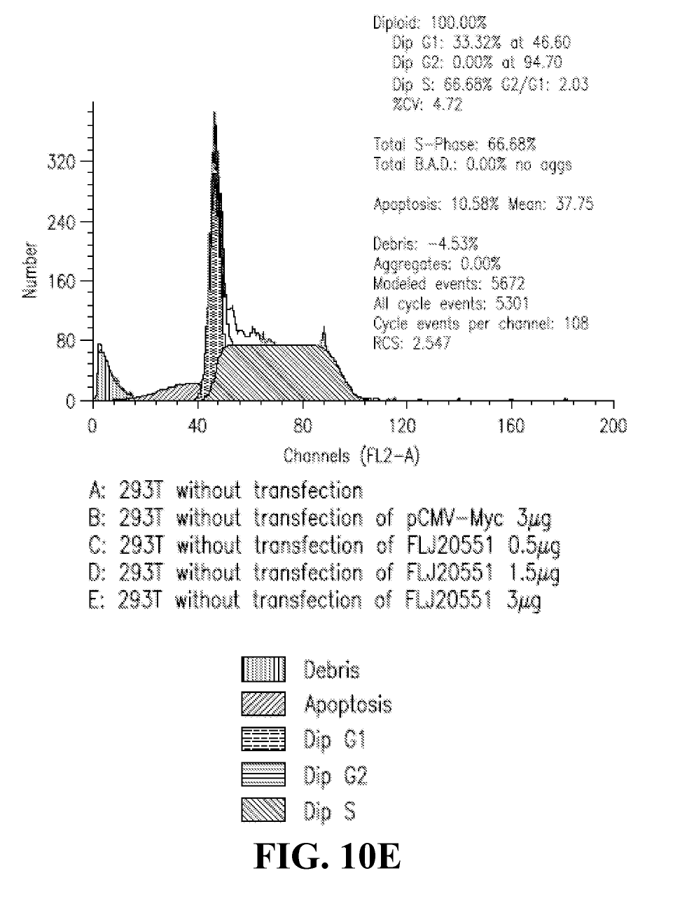

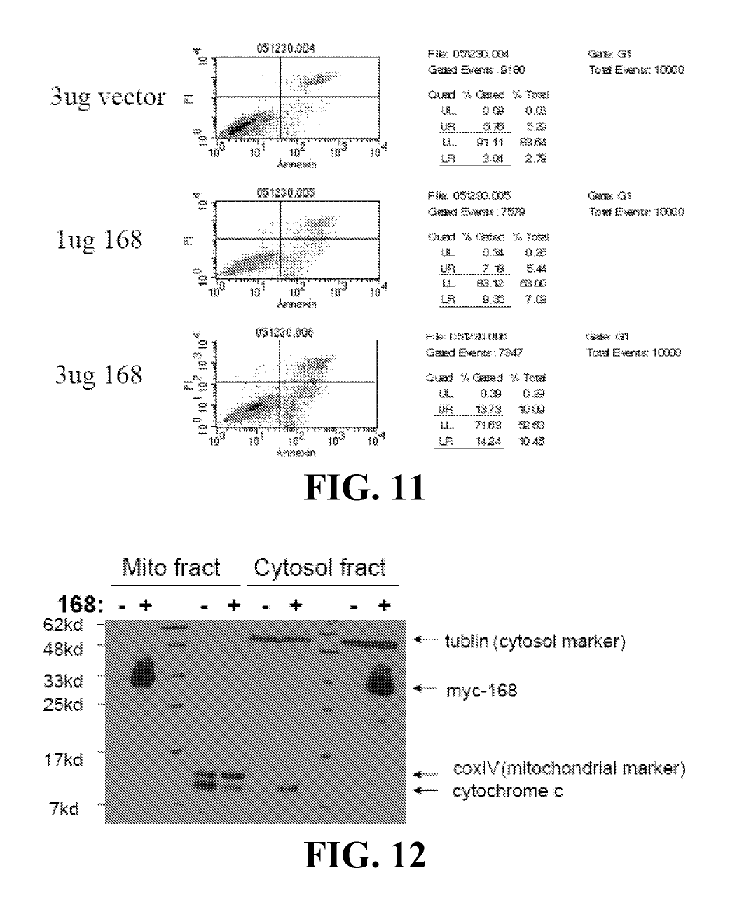

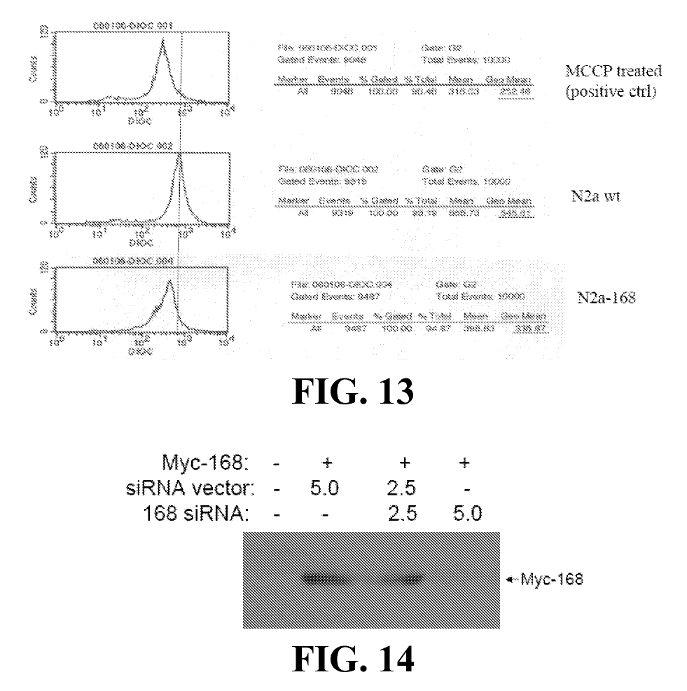

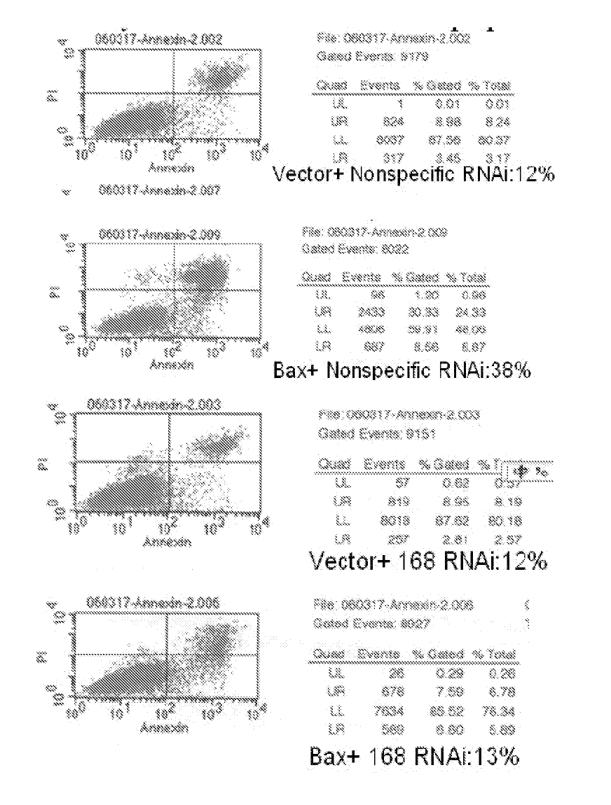

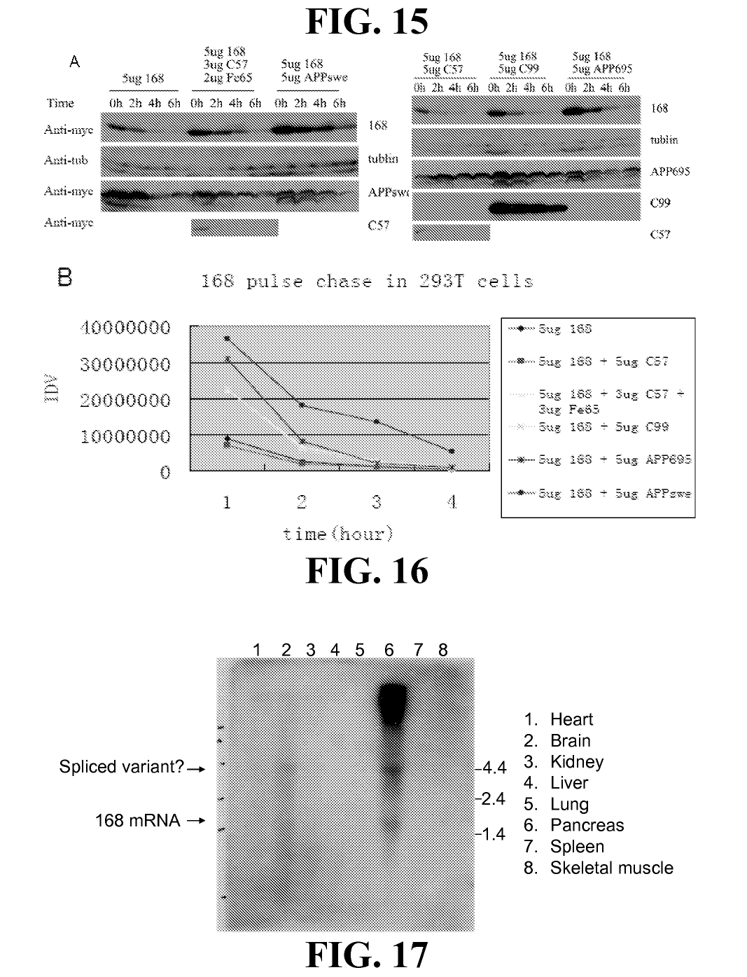

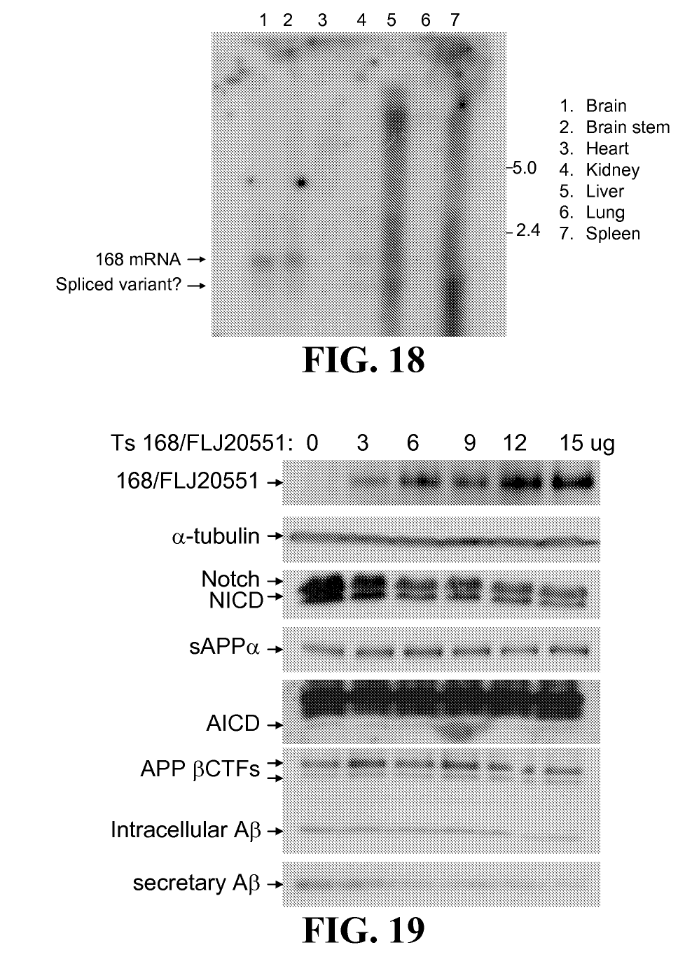

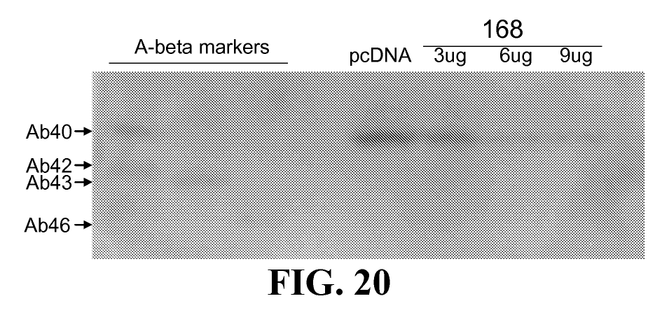

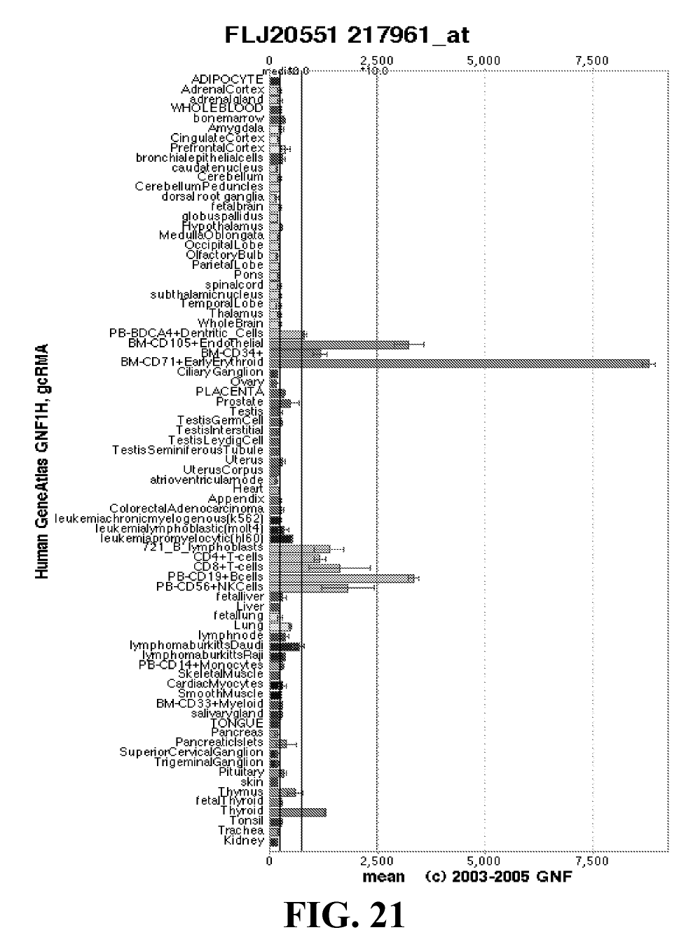

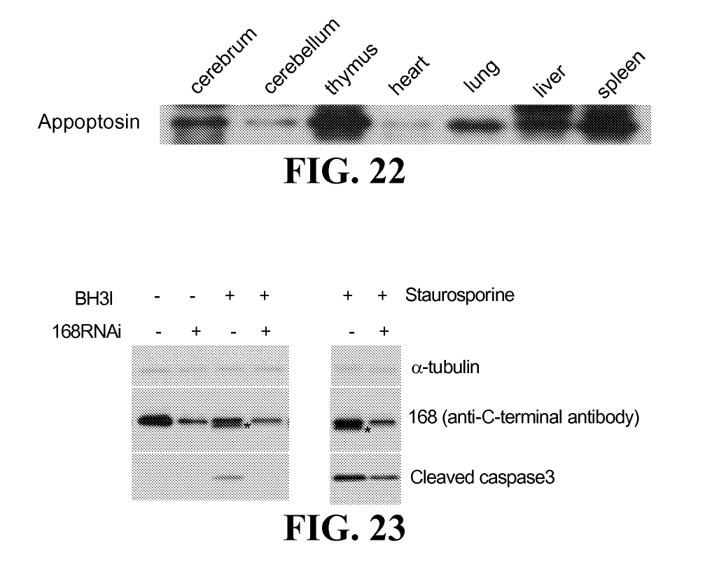

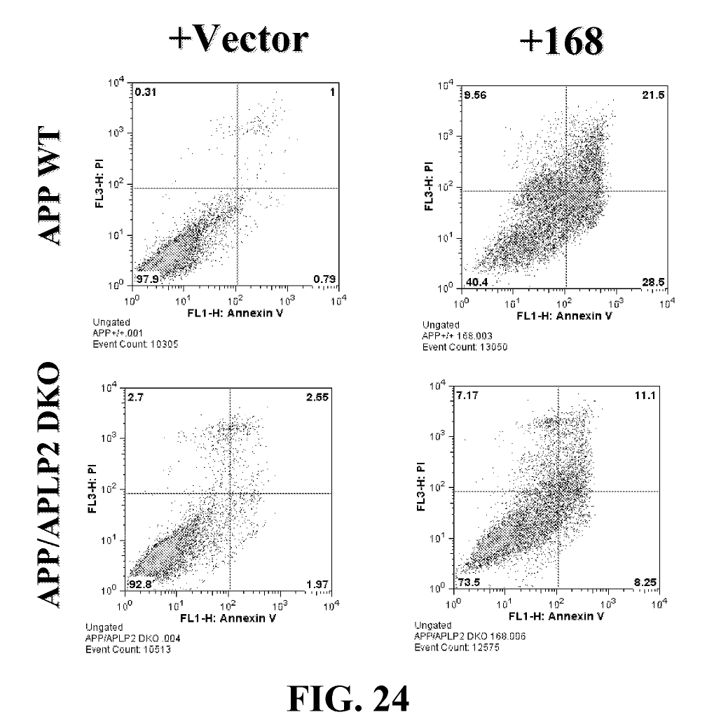

Examples of deletion mutants of Appoptosin are described in FIG. 2 and exemplified in FIGS. 5 and 6. It is understood that other such fragments or mutants of Appoptosin can be used in the disclosed compositions and methods and that one of skill in the art would be able to design and test such fragments or mutants using routine experimentation based on the guidance provided herein.

Thus, in some aspects, the disclosed polypeptide can comprise the amino acid sequence set forth in, for example, amino acids 118 to 264 of SEQ ID NO:2, amino acids 138 to 286 of SEQ ID NO:3, amino acids 138 to 286 of SEQ ID NO:4, amino acids 116 to 262 of SEQ ID NO:5, or amino acids 101 to 247 of SEQ ID NO:6.

Thus, the disclosed polypeptide can comprise an amino acid sequence having, for example, at least about 70, 75, 80, 85, 90, 95%, or 100% sequence identity to the amino acid sequence set forth in amino acids 118 to 264 of SEQ ID NO:2. The disclosed polypeptide can comprise an amino acid sequence having, for example, at least about 70, 75, 80, 85, 90, 95%, or 100% sequence identity to the amino acid sequence set forth in amino acids 138 to 286 of SEQ ID NO:3. The disclosed polypeptide can comprise an amino acid sequence having, for example, at least about 70, 75, 80, 85, 90, 95%, or 100% sequence identity to the amino acid sequence set forth in amino acids 138 to 286 of SEQ ID NO:4. The disclosed polypeptide can comprise an amino acid sequence having, for example, at least about 70, 75, 80, 85, 90, 95%, or 100% sequence identity to the amino acid sequence set forth in amino acids 116 to 262 of SEQ ID NO:5. The disclosed polypeptide can comprise an amino acid sequence having, for example, at least about 70, 75, 80, 85, 90, 95%, or 100% sequence identity to the amino acid sequence set forth in amino acids 101 to 247 of SEQ ID NO:6.

In other aspects, the disclosed polypeptide can comprise the amino acid sequence set forth in, for example, amino acids 264 to 304 of SEQ ID NO:2, amino acids 286 to 326 of SEQ ID NO:3, amino acids 286 to 326 of SEQ ID NO:4, amino acids 262 to 302 of SEQ ID NO:5, or amino acids 247 to 287 of SEQ ID NO:6.

Thus, the disclosed polypeptide can comprise an amino acid sequence having, for example, at least about 70, 75, 80, 85, 90, 95%, or 100% sequence identity to the amino acid sequence set forth in amino acids 264 to 304 of SEQ ID NO:2. The disclosed polypeptide can comprise an amino acid sequence having, for example, at least about 70, 75, 80, 85, 90, 95%, or 100% sequence identity to the amino acid sequence set forth in amino acids 286 to 326 of SEQ ID NO:3. The disclosed polypeptide can comprise an amino acid sequence having, for example, at least about 70, 75, 80, 85, 90, 95%, or 100% sequence identity to the amino acid sequence set forth in amino acids 286 to 326 of SEQ ID NO:4. The disclosed polypeptide can comprise an amino acid sequence having, for example, at least about 70, 75, 80, 85, 90, 95%, or 100% sequence identity to the amino acid sequence set forth in amino acids 262 to 302 of SEQ ID NO:5. The disclosed polypeptide can comprise an amino acid sequence having, for example, at least about 70, 75, 80, 85, 90, 95%, or 100% sequence identity to the amino acid sequence set forth in amino acids 247 to 287 of SEQ ID NO:6.

i. Amyloid Precursor Protein (APP)

Without wishing to be bound by theory, Appoptosin binds APP, thereby promoting apoptosis and inhibiting the production of amyloid beta. Thus, in some aspects, a candidate Appoptosin modulator disclosed herein can tested for the ability to bind APP or modulate the binding of Appoptosin to APP. Amyloid precursor protein (APP) is an integral membrane protein expressed in many tissues and concentrated in the synapses of neurons. Its primary function is not known, though it has been implicated as a regulator of synapse formation and neural plasticity. APP is best known as the precursor molecule whose proteolysis generates amyloid beta, a 39-42 amino acid peptide whose amyloid fibrillar form is the primary component of amyloid plaques found in the brains of Alzheimer's disease patients.

A number of distinct, largely independently folding structural domains have been identified in the APP sequence. The extracellular region, much larger than the intracellular region, is divided into the E1 and E2 domains; E1 contains several subdomains including a growth factor-like domain (GFLD), a metal-binding motif, and a serine protease inhibitor domain that is absent from the isoform differentially expressed in the brain. The E2 domain contains a coiled coil dimerization motif and may bind proteoglycans in the extracellular matrix.

APP undergoes extensive post-translational modification including glycosylation, phosphorylation, and tyrosine sulfation, as well as many types of proteolytic processing to generate peptide fragments. It is commonly cleaved by proteases in the secretase family; alpha secretase and beta secretase both remove nearly the entire extracellular domain to release membrane-anchored carboxy-terminal fragments that may be associated with apoptosis. It is cleavage by gamma secretase within the membrane-spanning domain that generates the amyloid-beta fragment; gamma secretase is a large multi-subunit complex whose components have not yet been fully characterized, but notably include presenilin, whose gene has been identified as a major genetic risk factor for Alzheimer's.

ii. Peptide Variants

As discussed herein there are numerous variants of the Appoptosin protein that are known and herein contemplated. In addition, to the known Appoptosin species variants there are derivatives of the Appoptosin proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:2 is set forth in SEQ ID NO:1. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

2. Nucleic Acids

The provided methods can also comprise administering a nucleic acid encoding Appoptosin, or a fragment or conservative variant thereof. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

i. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to, for example, Appoptosin as well as any other protein disclosed herein that are disclosed on Genbank, and these sequences and others are herein incorporated by reference in their entireties as well as for individual subsequences contained therein. Those of skill in the art understand how to resolve sequence discrepancies and differences and to adjust the compositions and methods relating to a particular sequence to other related sequences. Primers and/or probes can be designed for any sequence given the information disclosed herein and known in the art.

iii. Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

3. Internalization Sequence

The provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 3).

TABLE 3
Cell Internalization Transporters

| Name | Sequence | SEQ ID NO |
|---|---|---|
| Antp | 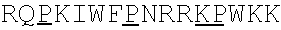 | (SEQ ID NO:14) |
| HIV-Tat | 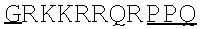 | (SEQ ID NO:15) |
| Penetratin | 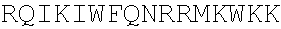 | (SEQ ID NO:16) |
| Antp-3A | 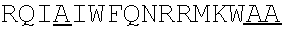 | (SEQ ID NO:17) |
| Tat | 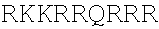 | (SEQ ID NO:18) |
| Buforin II | 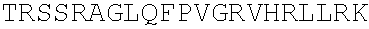 | (SEQ ID NO:19) |
| Transportan | 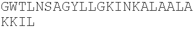 | (SEQ ID NO:20) |
| model amphipathic peptide (MAP) | 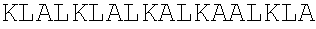 | (SEQ ID NO:21) |
| K-FGF | 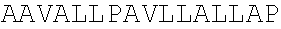 | (SEQ ID NO:22) |
| Ku70 | 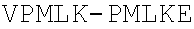 | (SEQ ID NO:23) |
| Prion | 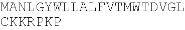 | (SEQ ID NO:24) |
| pVEC | 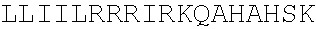 | (SEQ ID NO:25) |
| Pep-1 | 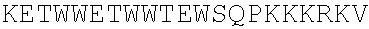 | (SEQ ID NO:26) |
| SynB1 | 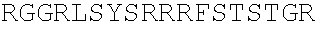 | (SEQ ID NO:27) |
| Pep-7 | 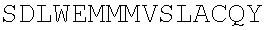 | (SEQ ID NO:28) |
| HN-1 | 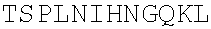 | (SEQ ID NO:29) |

BGSC (Bis-
Guanidinium-
Spermidine-
Cholesterol)

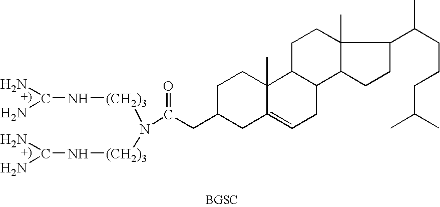

BGTC (Bis-
Guanidinium-Tren-
Cholesterol)

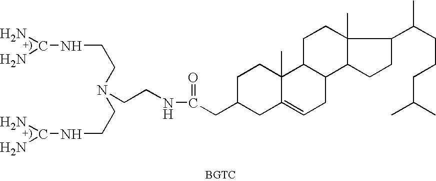

Thus, the provided polypeptide can further comprise the amino acid sequence SEQ ID NO:14, SEQ ID NO:15 (Bucci, M. et al. 2000. Nat. Med. 6, 1362-1367), SEQ ID NO:16 (Derossi, D., et al. 1994. Biol. Chem. 269, 10444-10450), SEQ ID NO:17 (Fischer, P. M. et al. 2000. J. Pept. Res. 55, 163-172), SEQ ID NO:18 (Frankel, A. D. & Pabo, C. O. 1988. Cell 55, 1189-1193; Green, M. & Loewenstein, P. M. 1988. Cell 55, 1179-1188), SEQ ID NO:19 (Park, C. B., et al. 2000. Proc. Natl. Acad. Sci. USA 97, 8245-8250), SEQ ID NO:20 (Pooga, M., et al. 1998. FASEB J. 12, 67-77), SEQ ID NO:21 (Oehlke, J. et al. 1998. Biochim. Biophys. Acta. 1414, 127-139), SEQ ID NO:22 (Lin, Y. Z., et al. 1995. J. Biol. Chem. 270, 14255-14258), SEQ ID NO:23 (Sawada, M., et al. 2003. Nature Cell Biol. 5, 352-357), SEQ ID NO:24 (Lundberg, P. et al. 2002. Biochem. Biophys. Res. Commun. 299, 85-90), SEQ ID NO:25 (Elmquist, A., et al. 2001. Exp. Cell Res. 269, 237-244), SEQ ID NO:26 (Morris, M. C., et al. 2001. Nature Biotechnol. 19, 1173-1176), SEQ ID NO:27 (Rousselle, C. et al. 2000. Mol. Pharmacol. 57, 679-686), SEQ ID NO:28 (Gao, C. et al. 2002. Bioorg. Med. Chem. 10, 4057-4065), or SEQ ID NO:29 (Hong, F. D. & Clayman, G. L. 2000. Cancer Res. 60, 6551-6556). The provided polypeptide can further comprise BGSC (Bis-Guanidinium-Spermidine-Cholesterol) or BGTC (Bis-Guanidinium-Tren-Cholesterol) (Vigneron, J. P. et al. 1998. Proc. Natl. Acad. Sci. USA. 93, 9682-9686). The preceding references are hereby incorporated herein by reference in their entirety for the teachings of cellular internalization vectors and sequences. Any other internalization sequences now known or later identified can be combined with a polypeptide disclosed herein.

4. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

5. Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

6. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

i. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as Appoptosin into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

c. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

d. Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

e. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

f. In Vivo/Ex Vivo

As described herein, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

ii. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b. Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

7. Administration

The provided compositions may be administered orally, rectally, intracisternally, intraventricular, intracranial, intrathecal, intra-articularly, intravaginally, parenterally (intravenously, intramuscularly, or subcutaneously), locally (powders, ointments, or drops), by intraperitoneal injection, transdermally, by inhalation or as a buccal or nasal spray. The exact amount of the therapeutic agent required will vary from subject to subject, depending on the age, weight and general condition of the subject, the severity of the disease that is being treated, the location and size of the tumor, the particular compounds used, the mode of administration, and the like. An appropriate amount may be determined by one of ordinary skill in the art using only routine experimentation given the teachings herein. Typical single dosages of agent range from 0.1-10,000 micrograms, preferably between 1 and 100 micrograms. Typical agent concentrations in a carrier range from 0.2 to 2000 nanograms per delivered milliliter. For injection into a joint, volumes of antibody and carrier will vary depending upon the joint, but approximately 0.5-10 ml, and preferably 1-5 ml, is injected into a human knee and approximately 0.1-5 ml, and preferably 1-2 ml into the human ankle.

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The composition can further comprise a pharmaceutically acceptable carrier. By “pharmaceutically acceptable” is meant a material that is not biologically or otherwise undesirable, which can be administered to an individual along with the selected substrate without causing significant undesirable biological effects or interacting in a deleterious manner with any of the other components of the pharmaceutical composition in which it is contained.

Depending on the intended mode of administration, the therapeutic agent can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. The compositions will include an effective amount of the selected substrate in combination with a pharmaceutically acceptable carrier and, in addition, may include other medicinal agents, pharmaceutical agents, carriers, or diluents. Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, ointments, powders, and solutions are also contemplated as being within the scope of this invention.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, S. M. Barge et al., “Pharmaceutical Salts,” J. Pharm. Sci., 1977, 66:1-19 which is incorporated herein by reference.)

In some aspects of the disclosed methods, the modulator of Appoptosin levels or activity can cross or readily pass through the blood-brain barrier, which permits, e.g., oral, parenteral or intravenous administration. Alternatively, the modulator can be modified or otherwise altered so that it can cross or be transported across the blood brain barrier. Many strategies known in the art are available for molecules crossing the blood-brain barrier, including but not limited to, increasing the hydrophobic nature of a molecule; introducing the molecule as a conjugate to a carrier, such as transferring, targeted to a receptor in the blood-brain barrier, or to docosahexaenoic acid etc.

In other aspects, the modulator can be administered intracranially or intraventricularly. In another aspect, osmotic disruption of the blood-brain barrier can be used to effect delivery of agent to the brain (Nilayer et al., 1995, Proc. Natl. Acad. Sci. USA 92:9829-9833). In yet another aspect, an agent can be administered in a liposome targeted to the blood-brain barrier. Administration of pharmaceutical agents in liposomes is known (see Langer, 1990, Science 249:1527-1533; Treat et al., 1989, in Liposomes in the Therapy of infectious Disease and Cancer, Lopez-Berestein and Fidler (eds.), Liss, New York, pp. 317-327 and 353-365). All of such methods are envisioned herein.

Other pharmaceutical compositions can be co-administered (sequentially or concurrently) with the disclosed modulator of Appoptosin levels or activity. Co-administration can be accomplished by administration of such other compositions in the same or in a different composition as those described above. In one aspect, an NSAID is co-administered, e.g., sulindac sulfide, flufenamic acid, ibuprofen, flurbiprofen, fenoprofen, mefenamic acid, indomethacin, carprofen or meclofenamic acid. In another aspect, a non-selective COX inhibitor is co-administered. In another aspect, a non-specific secretase inhibitor is co-administered. In another aspect, an antioxidant is co-administered. In specific aspects, the antioxidant is selected from the group consisting of vitamin E, vitamin C, curcumin, and Gingko biloba. In another aspect, an acetylcholinesterase inhibitor is co-administered.

8. Animal Models

In vivo animal models of APP processing into Aβ and of Aβ-related disorders such as Alzheimer's disease are well known to those of skill in the art. Such animal models can, for example, be used to further screen compounds identified via the methods presented herein. Alternatively, such models can be used to routinely validate compounds for efficacy in use in the modulation, prevention, treatment, and symptom amelioration methods of the present invention.

The wild-type guinea pig can be used as an animal model. For example, wild-type guinea pigs may be used as models for APP processing into Aβ, for Aβ production and/or accumulation of Aβ in brain. They are known to produce Aβ peptides that are immunologically identical to human Aβ. Therefore, the guinea pigs provide, for example, a model in which to examine perturbations in APP metabolism with and without drug treatment.

A mouse model of Alzheimer's disease can used, e.g., a transgenic mouse expressing human APP-Swedish (see, e.g., Borchelt et al., 1996, Neuron 17:1005-1013), which exhibits neuropathological symptoms similar to those of Alzheimer's disease patients. Moreover, a rat model of Alzheimer's disease can be used. A transgenic mouse model of Alzheimer's disease can be used, e.g., the Tg2576 transgenic mouse (U.S. Pat. No. 5,877,399). Such an animal model may be used to screen for compounds useful in the treatment of Alzheimer's disease due to its exhibition of, e.g., an altered level of human Aβ.

Models such as these can also be used to assess the efficacy of any potential therapeutic agents as disclosed herein. Generally, at least two groups of animals are used in the assay, with at least one group being a control group in which the administration vehicle is administered without the potential therapeutic agent.

Animal models can be employed that have an altered physiological regulation of the nervous system such that the animal or tissues derived from it can be utilized for screening of potential therapeutic agents and/or therapeutic regimens that act at the level of γ-secretase activity, especially at the level of competing (directly or indirectly) with ATP for activation of γ-secretase. Drugs that can reverse any of the defects exhibited by such an animal model act at some point in the catabolic pathway underlying the cleavage of APP to Aβ, and are thus of potential use therapeutically. Additionally, since some defects occur at the behavioral level, the alteration or modulation of these behavioral defects can have a high predictive value for therapeutic use in modification of such behaviors.

Also disclosed is a method for selecting a therapeutic agent for use in the treatment of a disorder, including but not limited to, an Aβ-related disorder, which comprises administering a modulator of Appoptosin levels or activity to an animal model, and measuring and/or determining the putative therapeutic agent's effect on any phenotypic characteristics which may be believed to be related to said disorder.

### E. METHODS OF SCREENING

Provided herein is a method of screening for an anti-amyloidogenic compound comprising administering a candidate agent to a sample comprising a cell having a nucleic acid encoding Appoptosin and monitoring the sample for changes in the expression of Appoptosin, wherein a detectable change in the expression of Appoptosin is an indication that the candidate compound can be used for treating or preventing Alzheimer's disease.

Also provided is a method of screening for a pro-apoptotic compound comprising administering a candidate agent to a sample comprising a cell having a nucleic acid encoding Appoptosin and monitoring the sample for changes in the expression of Appoptosin, wherein a detectable change in the expression of Appoptosin is an indication that the candidate compound can be used for treating or preventing cancer.

Examples of cells that can be used in the disclosed screening include, but are not limited to, Keratinizing Epithelial Cells, Wet Stratified Barrier Epithelial Cells, Exocrine Secretory Epithelial Cells, Hormone Secreting Cells, Epithelial Absorptive Cells (Gut, Exocrine Glands and Urogenital Tract), Metabolism and Storage cells, Barrier Function Cells (Lung, Gut, Exocrine Glands and Urogenital Tract), Epithelial Cells Lining Closed Internal Body Cavities, Ciliated Cells with Propulsive Function, Extracellular Matrix Secretion Cells, Contractile Cells, Blood and Immune System Cells, Sensory Transducer Cells, Autonomic Neuron Cells, Sense Organ and Peripheral Neuron Supporting Cells, Central Nervous System Neurons and Glial Cells, Lens Cells, Pigment Cells, Germ Cells, and Nurse Cells. Also included are any stem cells and progenitor cells of the cells disclosed herein, as well as the cells they lead to. Cells and cell types of interest produced in the disclosed method can be identified by reference to one or more characteristics of such cells. Many such characteristics are known, some of which are described herein.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of the target molecule should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that modulates Appoptosin. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of Appoptosin.

### F. METHOD OF DIAGNOSING OR MONITORING TREATMENT

Also provided is a method of identifying a subject having a leukemia or at risk of developing a leukemia, comprising detecting a level of Appoptosin in a blood or bone marrow cell of the subject that at least 10, 20, 30, 40, 50, 60, 70, 80, 90, 100, 110, 120, 130, 140, 150, 160, 170, 180, 190, or 200% greater than a reference standard.

Also provided is a method of monitoring the progress of a treatment for leukemia in a subject, comprising detecting the level of Appoptosin in a blood or bone marrow cell of the subject isolated before treatment and comparing it to the level of Appoptosin in a blood or bone marrow cell of the subject isolated after treatment, wherein a decrease in said Appoptosin levels after treatment is an indication of effective treatment.

### G. METHODS OF USING THE COMPOSITIONS AS RESEARCH TOOLS

The disclosed compositions can be used in a variety of ways as research tools. For example, the disclosed compositions, such as SEQ ID NOs:1-6 can be used to study the interactions between Appoptosin and APP, by for example acting as inhibitors of binding.

The compositions can be used for example as targets in combinatorial chemistry protocols or other screening protocols to isolate molecules that possess desired functional properties related to Appoptosin and APP.

### H. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. For example, disclosed are nucleic acids in SEQ ID NO:1. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO:1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO:1, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO:1 and a sequence controlling the expression of the nucleic acid.

Disclosed are nucleic acid molecules produced by the process comprising linking in an operative way a nucleic acid molecule comprising a sequence encoding a peptide set forth in SEQ ID NO:2 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO:2 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO:2, wherein any change from the SEQ ID NO:2 are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### I. SPECIFIC EMBODIMENTS

Provided is a method of treating or preventing Alzheimer's disease in a subject comprising administering to the subject a therapeutically effective amount of a modulator of Appoptosin levels or activity.

Also provided is a method of treating a subject at risk for Alzheimer's disease comprising administering to the subject a modulator of Appoptosin levels or activity.

Also provided is a method of slowing the onset of symptoms of Alzheimer's disease in a subject comprising administering to the subject a therapeutically effective amount of a modulator of Appoptosin levels or activity.

Also provided is a method of reversing the symptoms of Alzheimer's disease in a subject comprising administering to the subject a therapeutically effective amount of a modulator of Appoptosin levels or activity.

Also provided is a method of ameliorating the symptoms of Alzheimer's disease in a subject comprising administering to the subject a therapeutically effective amount of a modulator of Appoptosin levels or activity.

The subject can have been diagnosed with Alzheimer's disease. The composition can be administered to the subject systemically. The composition can be administered to the subject intracranially. The modulator of Appoptosin levels can be an Appoptosin polypeptide. The polypeptide can comprise the amino acid sequence set forth in SEQ ID NO:2. The polypeptide can comprise an amino acid sequence having at least 70, 75, 80, 85, 90, 95% sequence identity to the amino acid sequence set forth in SEQ ID NO:2.

The modulator of Appoptosin levels can be a nucleic acid encoding an Appoptosin polypeptide. The nucleic acid can comprise the nucleic acid sequence set forth in SEQ ID NO:1. The nucleic acid can comprise an nucleic acid sequence having at least 70, 75, 80, 85, 90, 95% sequence identity to the nucleic acid sequence set forth in SEQ ID NO:1. The nucleic acid can hybridize under stringent conditions to a nucleic acid consisting of the nucleic acid sequence set forth in SEQ ID NO:1.

Also provide is a method of inducing apoptosis in a target cell comprising contacting the target cell with a therapeutic amount of a modulator of Appoptosin levels or activity. Also provided is a method of treating or preventing cancer in a subject comprising administering to the subject a therapeutically effective amount of a modulator of Appoptosin levels or activity.

Also provided is a method of screening for an anti-amyloidogenic compound comprising administering a candidate agent to a sample comprising a nucleic acid encoding Appoptosin and monitoring the sample for changes in the expression of Appoptosin, wherein a detectable change in the expression of Appoptosin is an indication that the candidate compound can be used for treating or preventing Alzheimer's disease.

Also provided is a method of screening for a pro-apoptotic compound comprising administering a candidate agent to a sample comprising a nucleic acid encoding Appoptosin and monitoring the sample for changes in the expression of Appoptosin, wherein a detectable change in the expression of Appoptosin is an indication that the candidate compound can be used for treating or preventing cancer.

Also provided is a method of making an agent that modulates Appoptosin, comprising administering a candidate agent to a sample comprising a nucleic acid encoding Appoptosin, detecting a change in the expression of Appoptosin, and making the candidate agent.

Also provided is an expression vector comprising a nucleic acid encoding Appoptosin operably linked to an tissue specific expression control sequence. For example, the vector can selectively expresses Appoptosin in the brain.

### J. EXAMPLES

### 1. Example 1

### A Study of AICD and Interacting Protein by Yeast Towo-Hybrid

i. Materials and Methods

MATCH MAKER GAL4 Two-Hybrid System 3 was purchased from Clontech, restriction enzymes from Takara, 3-AT (3-amino-1,2,4-triazole), PEG3350 (Polyethylene glycol), ONPG (o-nitrophenyl-D-galactopyranoside) and X-GAL (5-bromo-4-chloro-3-indolyl-β-D-galactopyranoside) from Sigma.

Cells and major reagents: HEK293T was maintained in DMEM (GIBCO) supplemented with 10% fetal bovine serum. Protease inhibitors and ECL were from Roche and protein-A Sepharose from Zymed.

Construction and Expression of Bait Plasmid: The template chosen is an eukaryotic expression vector containing the full length human APP. The primers used are

| | |
|---|---|
| 5′-GCGGAATTCGGTGTTGTCATAGCG-3 | (SEQ ID NO:7) |
| and | |
| 5′-CGCGGATCCCTAGTTCTGCATCTG-3′. | (SEQ ID NO:8) |

The intracellular end of human APP was amplified, i.e., AID, was amplified. In order to completely expose AID, 5 amino acids of Aβ and GGGGSGGGGSGGGGS (SEQ ID NO:9) were extended in front of AID. The PCR product was recovered and purified. It was then inserted into protein expression vector pGBKT7 for identification by means of sequencing. The successfully constructed vector is named pGBKT7-AID. Positively identified pGBKT7-AID was transferred into yeast AH109, and then cultured on a SD/-Trp plate. Clones were picked and protein was extracted following the method described in Molecular Cloning: A Laboratory Manual” (Third Edition) to determine the expression situation.

Detection of Autonomous Activation of Bait Protein: The successfully constructed plasmid pGBKT7-AID was transformed onto yeast AH109 alone. Autonomous activation was observed on a SD/-Leu/-Trp/-His culture plate at 3-AT concentrations of 0, 10, 15, 20 and 25 mM. The appropriate 3-AT concentration was chosen for screening. The prey plasmids were extracted from yeast, amplified in DH5a and identified by restriction enzyme digestion and DNA sequencing.

Screening and Identification of Positive Clones: Yeast expression vector pGBKT7 contained the autocrine gene of Trp (tryptophan) and the DNA binding domain of GAL4. pACT2 contained the autocrine gene of Leu (leucine) and the DNA activation structure domain. As a result, the bait plasmid and the sDNA library built on pACT2 could grow on SD/-Trp and SD/-Leu culture plates after they were transformed into AH1109. Upon completion of co-transformation, growth could take place on a SD/-Trp/-Leu culture plate. If there was mutual interaction between the bait protein and proteins expressed in the library, the DNA binding domain would bind with its activation domain on GAL4 to activate the expression of the LacZ gene downstream to enable the clone to grow on a SD/-Trp/-Leu/-His culture plate. This was defined as a positive clone. As far as procedures are concerned, the operating manual of Clonetech was followed. Yeast positive clone plasmid was extracted. It was used to transfect Escherichia coli DH5α competent cells by way of electroporation. Plasmid was then extracted, digested in enzyme, categorized and sequenced. The results were compared and analyzed against information available in GenBank.

Construction of Full Length cDNA and Bait Protein Eukaryotic Expression Plasmid:

RPS21: Positive clone plasmid was used as the template and

| | |
|---|---|
| 5′-GCGGAATTCGGATGCAGAACGACGCC-3′ | (SEQ ID NO:30) |
| and | |
| 5′-CGCCTCGAGTCAAAAGTTCTTTGA-3′ | (SEQ ID NO:31) |

were used as primers to amplify the gene of the protein needed. The product was inserted into plasmid pCMV-Myc for identification by way of sequencing.

FLJ20551: Full length cDNA was amplified from cDNA in Hela cells by using

(SEQ ID NO:10)

5′-GCGAGATCTCTATGATTCAGAACTCACGTCCG-3′
and

SEQ ID NO:11)

5′-CGCGGTACCTCAGGACTTCAGGCCCATCTTGG-3′

as primers. The product was inserted into plasmid pCMV-Myc and identified by sequencing. Successfully constructed plasmids were named pCMV-Myc-RPS21 and pCMV-Myc-FLJ20551, respectively.

Construction of bait protein expressing plasmid in mammalian cells: cDNA fragment of AID was amplified by PCR from pGBKT7-AID by using

| | |
|---|---|
| 5′-GCGGAATTCGGTGTTGTCATAGCG-3′ | (SEQ ID NO:7) |
| and | |
| 5′-CGCGGATCCCTAGTTCTGCATCTG-3′ | (SEQ ID NO:8) |

as primers. The product was inserted into vector pCMV5-HA to obtain plasmid pCMV5-HA-AID for identification by sequencing.

Construction of Full Length RPS21 and FLJ20551 in Yeast Expression Vector pGADT7: RPS21 target fragment was cleaved off from pCMV-Myc-RSP21 with EcoR I and Xho I and then inserted it into pGADT7. For FLJ20551, a fragment was amplified from pCMV-Myc-FLJ20551 by using

| | |
|---|---|
| 5′-GCGGGATCCTGATTCAGAACTCACGT-3′ | (SEQ ID NO:12) |
| and | |
| 5′-CGCCTCGAGTCAGGACTTCAGGCCCA-3′ | (SEQ ID NO:13) |

as primers. It was then inserted into pGADT7, digested with enzyme and identified by sequencing. The successfully constructed plasmids were named pGADT7-RPS21 and pGADT7-FLJ20551.

Quantitative Analysis of β-galactosidase Activity: Yeast co-transfected positive clones grown on the plate were picked and cultured further in a SD/Leu/-Trp liquid culture medium at 30° C. overnight. It was transferred to a YPD culture medium the next morning to grow until A_{600 nm}=1.0˜1.5. Then, after rinsing the product with Z buffer (Na₂HPO₄.7H₂O, 16.1 g/L; NaH₂PO₄.H₂O, 5.50 g/L; KCl, 0.75 g/L; MgSO₄.7H₂O, 0.246 g/L; pH adjusted to 7.0 with NaOH), it underwent three freeze and thaw cycles in liquid nitrogen. After adding o-nitrophenyl-β-galactoside (ONPG) as the substrate, the temperature was kept at 30° C. Upon completion of staining, the reaction was terminated with Na₂CO₃. The solution was centrifuged and the supernatant was taken to measure its absorbance at 420 nm to calculate its α-galactosidase activity. Each experiment was repeated three times. The intensity of interaction was quantitatively determined based on β-galactosidase activity.

Immuno-Coprecipitation: Constructed plasmids pCMV-Myc-RPS21 and pCMV5-HA-AID were co-transfected into 293T cells using the calcium phosphate technique. After incubating for 48 hours, approximately 750 μL of cell lysate (10 mmol/L Tris pH 7.5, 150 mmol/L NaCl, 1 mmol/L EDTA, 0.5% NP-40, and protease inhibitor) was added. Cells were scraped off and mixed thoroughly at 4° C. to ensure that cells were lysed fully. It was centrifuged at 14,000 r/min and the supernatant was taken for further processing. Antibody 369 (used to identify AID) and 25 μL of Sepharose were added. The solution was cultured at 4° C. for 6˜10 hours. It was then centrifuged at 10,000 r/min for 10 seconds, and then rinsed with TNE (10 mmol/L Tris pH 7.5, 150 mmol/L NaCl, 1 mmol/L EDTA and 0.5% NP-40) four times. Upon completion, fluid was carefully removed, and 2× sample loading buffer was added. The solution was boiled at 100° C. for 5˜10 minutes. The solution was aspirated and analyzed by 10% SDS-PAGE. After proteins were separated by gel electrophoresis, they were transferred to nitrocellulose membranes. Protein expression and interaction were tested by Western blotting.

ii. Results

Construction of Bait Plasmid and Detection of Autonomous Activation: In order to find new proteins that interact with AID, bait plasmid pGBKT7-AID was constructed. After verifying its accuracy by means of sequencing, it was transformed into yeast AH109 using the Li—Ac technique. Its expression was found to be normal. In order to eliminate false positives caused by autonomous activation of the bait protein, the successfully constructed plasmid pGBKT7-AID was used to transfect yeast AH109 by itself. Its autonomous activation effect on SD/-Leu/-Trp auxotrophic plates at a series of 3-AT concentrations, including 0, 10, 15, 20 and 25 mmol/L. 3-AT is widely used to inhibit autonomous activation of bait protein. These results showed that pGBKT7-AID could not grow on SD/-Leu/-Trp auxotrophic plate when the concentration of 3-AT was at 15 mmol/L. This indicates that there is no autonomous activation of pGBKT7-AID at this concentration. This condition was used for library screening.

Yeast Two-Hybrid Screening: Based on Clonetech's operating manual, bait plasmid pGBKT7-AID and library of human embryo brain cDNA on pACT2 were co-transformed into yeast AH109. The product was allowed to grow on a SD/-Trp/-Leu/-His auxotrophic culture plate to yield 800 clones. After screening with 150 mmol/L 3-AT, 120 clones remained. Stronger interacting clones were further selected by means of staining with α-galactosidase. Plasmid was extracted and digested with enzyme to examine fragment sizes. Furthermore, identical clones were eliminated. Then, these clones were transformed back to yeast to detect for autonomous activation again. The last 48 clones were sequenced.

Sequence Analysis: Sequences measured in this study were compared with data available in GenBank to find the full length gene corresponding to this cDNA. Clone #23 is ribosomal protein RPS21. The full length of its open reading frame is 252 bp, which is completely contained in the positive clone obtained in this study. It matches at 100%. Another clone, #168, is the new protein FLJ20551, not previously described. The clone obtained includes the C terminus of the opening reading frame of the gene fragment. It is approximately ⅛ of its full length. It also matches at 100%.

Quantitative Analysis of β-galactosidase Activity: In order to verify the specificity and strength of interaction between RPS-21 and AID and between FLJ20551 and AID, these two proteins in full length were cloned into yeast expression vector pGADT7. Plasmids obtained in this study as described in Section 1.2.5 were identified by enzyme digestion. Finally, these plasmids were positively identified by way of sequencing.

pGADT7 contains the GAL4 activation domain: When pGADT7-RPS21 and pGADT7-FLJ20551 were individually co-transfected into yeast AH109 with pGBKT7-AID, if RPS21 or FLJ20551 interacts with AID, then the activation domain of GAL4 can be carried into the AID-containing pGBKT7. Furthermore, it can interact with the GAL4 DNA binding domain expressed by pGBKT7 to activate the LacZ gene downstream to secrete β-galactosidase. It is possible to detect the intensity of interaction between two proteins by using ONPG as the substrate. Quantitative analysis of β-galactosidase activity showed that despite the fact that the effect of co-transfection with pACT2-RPS2, a fragment of RPS21, and pGBKT7-AID was not significantly different from that of negative control (which is pACT2-RPS21 and pGBKT7), there was apparent interaction between full length RPS21 and AID (as shown in FIG. 3). Either in the form of a fragment or in full length, the interaction between FLJ20551 and the bait protein was found to be fairly intense. These results indicate that interaction between RPS21 and AID and between FLJ20551 and AID are specific in yeast, as illustrated in the β-galactosidase experiment.

Results of Immuno-Coprecipitation: In order to further verify the interaction between RPS21 and AID in mammalian cells, RPS21 and AID were transformed into HEK293T cells to study their mutual interaction at mammalian cell level. These results showed that RPS21 could be expressed quite normally in mammalian cells. Upon co-transfection with AID, obvious interaction was detected This illustrates that interaction between RPS21 and AID in mammals is also specific.

K. Sequences

1. SEQ ID NO:1 (human Appoptosin; FLJ20551)

| | |
|---|---|
| 1 | gaagcgaaat ctccccttct acagagtccc tccggcgctt cctccacccc gggatacaca |
| 61 | gaacctcatc tcctacggtg ctgaagcctg cagcagggca ggatgggcag gagagcagag |
| 121 | ccgcggagtc tgcggcgcgg gtgaagagcg gcgcgtaatt cccgcagcaa gattgttccg |
| 181 | cgcccgcagc ccctggacta gcaggatccg aaccccggcg gctgcgtgct tataggcgca |
| 241 | gacgtcagag agcccgcggc ttaaagcgcg tcgcctggct agcgccaccc cctagccttc |
| 301 | ttcaaggcct ccagggctgg gcccaagcgc ccgtcgacgg caccctgggc ccagaggact |
| 361 | cgcgggcctc atctccaatg attcagaact cacgtccgtc gctgctgcaa ccccaagatg |
| 421 | tcggagacac ggtggaaacg cttatgttac atccggtgat caaggctttc ctgtgtggct |
| 481 | ccatcagtgg gacctgctct accctccttt tccaacctct ggatctcctt aaaacacgcc |
| 541 | tacaaaccct ccagccctca gatcatgggt ctagacgtgt tgggatgttg gctgtactct |
| 601 | tgaaggtggt tcgcacggag agtcttttgg gcctttggaa agggatgtcc ccttccattg |
| 661 | tgagatgtgt ccctggcgtt ggaatctact ttggcactct ctactctttg aagcagtatt |
| 721 | tcttgcgagg ccatccccca accgccctgg agtcagtcat gctgggggtg ggctctcgct |
| 781 | ctgttgcagg ggtctgtatg tcacctatca ctgtaatcaa gacgcgctat gagagtggga |
| 841 | aatatggcta tgagagtatc tacgctgccc tgaggagcat ctatcacagt gaggggcacc |
| 901 | ggggcctctt cagtggcctg acagcaactc tccttcgaga tgcgcccttc tcaggaatct |
| 961 | acctgatgtt ttacaaccag accaaaaata tagtgcctca tgaccaggtg gatgcaaccc |
| 1021 | ttattcctat tacaaatttc agctgtggga tatttgctgg tattctggcc tcactggtaa |
| 1081 | ctcaacctgc gggtgttatc aaaactcata tgcagcttta tccactgaag tttcaatgga |
| 1141 | ttggccaagc agtgacactt attttcaaag actatggact acgtggcttc ttccaaggtg |
| 1201 | gcatcccccg agccctccgc agaactctaa tggcagcaat ggcgtggacg gtgtatgaag |
| 1261 | agatgatggc caagatgggc ctgaagtcct gaccaagaga ggactgggaa cgggtgaaat |
| 1321 | ctgttgccct gcctggtttc tgccaagggc tgctgcttct tactattctg cagtaagatg |
| 1381 | aagtcctacc tggaaaacca ggcagaaatt gtgttgcctt tgccttcagt aatcccctta |
| 1441 | aggagaaaat atatggacct gatttcagcc ttcagaatct ccaaaagagg agtcatcaat |
| 1501 | tcatagagca cactagggtg ttaggagaga gctttgcata ctctgagagg ctacttggaa |
| 1561 | aggcattttc ccaggagagc tctgtcaggt ggctgcgctt cagccccacc cctacaccac |
| 1621 | agggtctcct tgggtatgtt cttgggcaag caatcacaaa gccagagaag ctgtaagctg |
| 1681 | cctgccgggc ctgaggagct ccaaccaggg aagactggat gtgaggagag gagtcactgt |
| 1741 | caccaggtca cagactgact gaggtgatgg taggatgagg aggaacagat gcccttcttt |
| 1801 | aattggttct cagttaactt ctcagaggct ctggagaacg ggacagtggc tttctagcct |
| 1861 | ctgaatgttc caaataaaat tttttggtct tggcccctgt actgttttac ctctaaattc |
| 1921 | tggcattttt ttttccctgc aattaaagtg ctgtacttta tttattgaac tttatcattg |
| 1981 | aaagctgttt ggaaaaccta aaaaaaaaaa aaaaaa |

2. SEQ ID NO:2 (human Appoptosin; FLJ20551)
MIQNSRPSLLQPQDVGDTVETLMLHPVIKAFLCGSISGTCSTLLFQPLDLLKTRLQTLQPSDHGSRRVGM
LAVLLKVVRTESLLGLWKGMSPSIVRCVPGVGIYFGTLYSLKQYFLRGHPPTALESVMLGVGSRSVAGVC
MSPITVIKTRYESGKYGYESIYAALRSIYHSEGHRGLFSGLTATLLRDAPFSGIYLMFYNQTKNIVPHDQ
VDATLIPITNFSCGIFAGILASLVTQPAGVIKTHMQLYPLKFQWIGQAVTLIFKDYGLRGFFQGGIPRAL
RRTLMAAMAWTVYEEMMAKMGLKS
3. SEQ ID NO:3 (mouse Appoptosin; AAH10801)
MGVSAEPRSLSVAGAGLASPVIEKARSALLQSQDVEDTVETLMLHPVIKAFLCGSISGTCSTLLFQPLDL
LKTRLQALQPSDLGPRRVGMLAVFLKVVRTESLLGLWKGMSPSIVRCVPGVGIYFGTLYSSKQYFLRGHP
PTALESVILGMGSRSVAGVCMSPITVIKTRYESGTYSYESIYAALRSIYCSEGHRGLFRGLTATLLRDAP
FSGLYLMFYSQTRTAVLHGTAQLDAALIPLINFSCGIFAGVLASLVTQPADVIKTHMQLSPVKFQWIGQA
ATLIFKNHGLRGFFHGSVPRALRRTLMAAMAWTVYEEMMARMGLKS
4. SEQ ID NO:4 (rat Appoptosin; AAH99762)
MGVSAEPRSLSVARAGLASPVIEKARSALLPSQDVEDTVETLMLHPVIKAFLCGSISGTCSTLLFQPLDL
LKTRLQTLQPSDVGPRRVGMLSVFLKVVRTETLLGLWKGMSPSIVRCVPGVGIYFGTLYSSKQYFLRGHP
PTALESVILGMGSRSVAGVCMSPITVVKTRYESGAYSYESVYAALRSIYCSEGSRGLFRGLTATLLRDAP
FSGLYLMFYSQTRATVLHGADELDAALMPLVNFSCGVFAGILASLVTQPADVIKTHMQLSTVKCQCIGQV
ATLILKTHGLRGFFHGSVPRALRRTLMAAMAWTVYEEMMAKMGLKS
5. SEQ ID NO:5 (xenopus Appoptosin; AAH77266)
MSNALVVAGDSLVPSNRVSQMHPVFKAFVCGSLSGTCSTLLFQPLDLVKTRIQAHQLSASAAGSRPRMLN
LLIKVVRNENILGLWKGVSPSFLRCIPGVGLYFSTLYTLKHHFFSERDPKPLESVMLGAGSRTVAAVCML
PFTVVKTRYESGKYGYNSVYGALKAIYKTEGPRGLFSGLTATLMRDAPFSGIYLMFYTRAKKLAPHDQID
PLFSPVLNFSCGIVAGILASVATQPADVIKTHMQLANEKYHWTGKVALNIYRTQGLTGFFQGGVPRALRR
TLMAAMAWTVYEQMMEKMGLKS
6. SEQ ID NO:6 (zebrafish Appoptosin; XP_700700)
MEVALAHPALKAFMCGSLSGTCSTLLFQPLDLVKTRLQTLQNNMHPGAPKVGMITVLFNVIRTEKLLGLW
KGVSPSFMRCIPGVGIYFSTFYSLKQHYFQEGSPSAGEAVLLGAGARCVAGVAMLPFTVIKTRFESGRYN
YISVAGALKSVCQNEGPKALYSGLTATLLRDAPFSGIYVMFYSQAKKALPQEISSSSIAPLVNFGCGVVA
GILASLATQPADVIKTHMQVSPALYPKTSDAMRHVYVKHGLSGFFRGAVPRSLRRTLMAAMAWTVYEQLM
ARMGLKS
7. SEQ ID NO:7
GCGGAATTCGGTGTTGTCATAGCG
8. SEQ ID NO:8
CGCGGATCCCTAGTTCTGCATCTG
9. SEQ ID NO:9
GGGGSGGGGSGGGGS (amino acids)
10. SEQ ID NO:10
GCGAGATCTCTATGATTCAGAACTCACGTCCG
11. SEQ ID NO:11
CGCGGTACCTCAGGACTTCAGGCCCATCTTGG
12. SEQ ID NO:12
GCGGGATCCTGATTCAGAACTCACGT
13. SEQ ID NO:13
CGCCTCGAGTCAGGACTTCAGGCCCA
14. SEQ ID NO:14
RQPKIWFPNRRKPWKK
15. SEQ ID NO:15
GRKKRRQRPPQ
16. SEQ ID NO:16
RQIKIWFQNRRMKWKK
17. SEQ ID NO:17
RQDIAIWFQNRRMKWAA
18. SEQ ID NO:18
RKKRRQRRR
19. SEQ ID NO:19
TRSSRAGLQFPVGRVHRLLRK
20. SEQ ID NO:20
GWTLNSAGYLLGKINKALAALAKKIL
21. SEQ ID NO:21
KLALKLALKALKAALKLA
22. SEQ ID NO:22
AAVALLPAVLLALLAP
23. SEQ ID NO:23
VPMLK-PMLKE
24. SEQ ID NO:24
MANLGYWLLALFVTMWTDVGLCKKRPKP
25. SEQ ID NO:25
LLIILRRRIRKQAHAHSK
26. SEQ ID NO:26
KETWWETWWTEWSQPKKKRKV
27. SEQ ID NO:27
RGGRLSYSRRRFSTSTGR
28. SEQ ID NO:28
SDLWEMMMVSLACQY
29. SEQ ID NO:29
TSPLNIHNGQKL
30. SEQ ID NO:30
GCGGAATTCGGATGCAGAACGACGCC
31. SEQ ID NO:31
5′-CGCCTCGAGTCAAAAGTTCTTTGA

What is claimed is:

1. A method of treating a subject having or at risk for developing Alzheimer's disease, comprising administering to the subject a therapeutically effective amount of a modulator of Appoptosin levels or activity.

2. The method of claim 1, wherein the subject has been diagnosed with Alzheimer's disease.

3. The method of claim 1, wherein the composition is administered to the subject systemically.

4. The method of claim 1, wherein the composition is administered to the subject intracranially.

5. The method of claim 1, wherein the modulator of Appoptosin levels is an Appoptosin polypeptide.

6. The method of claim 5, wherein the polypeptide comprises the amino acid sequence set forth in SEQ ID NO:2, 3, 4, 5, or 6.

7. The method of claim 5, wherein the polypeptide comprises the amino acid sequence set forth in amino acids 118 to 264 of SEQ ID NO:2, amino acids 138 to 286 of SEQ ID NO:3, amino acids 138 to 286 of SEQ ID NO:4, amino acids 116 to 262 of SEQ ID NO:5, or amino acids 101 to 247 of SEQ ID NO:6.

8. The method of claim 5, wherein the polypeptide comprises an amino acid sequence having at least 95% sequence identity to the amino acid sequence set forth in SEQ ID NO:2.

9. The method of claim 5, wherein the polypeptide comprises an amino acid sequence having at least 95% sequence identity to the amino acid sequence set forth in amino acids 118 to 264 of SEQ ID NO:2.

10. The method of claim 5, wherein the polypeptide comprises an amino acid sequence having at least 95% sequence identity to the amino acid sequence set forth in SEQ ID NO:3.

11. The method of claim 5, wherein the polypeptide comprises an amino acid sequence having at least 95% sequence identity to the amino acid sequence set forth in amino acids 138 to 286 of SEQ ID NO:3.

12. The method of claim 5, wherein the polypeptide comprises an amino acid sequence having at least 95% sequence identity to the amino acid sequence set forth in SEQ ID NO:4.

13. The method of claim 5, wherein the polypeptide comprises an amino acid sequence having at least 95% sequence identity to the amino acid sequence set forth in amino acids 138 to 286 of SEQ ID NO:4.

14. The method of claim 5, wherein the polypeptide comprises an amino acid sequence having at least 95% sequence identity to the amino acid sequence set forth in SEQ ID NO:5.

15. The method of claim 5, wherein the polypeptide comprises an amino acid sequence having at least 95% sequence identity to the amino acid sequence set forth in amino acids 116 to 262 of SEQ ID NO:5.

16. The method of claim 5, wherein the polypeptide comprises an amino acid sequence having at least 95% sequence identity to the amino acid sequence set forth in SEQ ID NO:6.

17. The method of claim 5, wherein the polypeptide comprises an amino acid sequence having at least 95% sequence identity to the amino acid sequence set forth in amino acids 101 to 247 of SEQ ID NO:6.

18. The method of claim 1, wherein the modulator of Appoptosin levels is a nucleic acid encoding an Appoptosin polypeptide.

19. The method of claim 18, wherein the nucleic acid comprises the nucleic acid sequence set forth in SEQ ID NO:1.

20. The method of claim 18, wherein the nucleic acid comprises an nucleic acid sequence having at least 95% sequence identity to the nucleic acid sequence set forth in SEQ ID NO:1.

21. The method of claim 18, wherein the nucleic acid hybridizes under stringent conditions to a nucleic acid consisting of the nucleic acid sequence set forth in SEQ ID NO:1.

22. A method of inducing apoptosis in a target cell comprising contacting the target cell with a therapeutic amount of a modulator of Appoptosin levels or activity.

23. A method of treating or preventing cancer in a subject comprising administering to the subject a therapeutically effective amount of a modulator of Appoptosin levels or activity.

24. An expression vector comprising a nucleic acid encoding Appoptosin operably linked to an tissue specific expression control sequence.

25. The expression vector of claim 24, wherein the vector selectively expresses Appoptosin in the brain.

26. A method of screening for an anti-amyloidogenic compound comprising administering a candidate agent to a sample comprising a cell having a nucleic acid encoding Appoptosin and monitoring the sample for changes in the levels of Appoptosin, wherein a detectable change in the levels of Appoptosin is an indication that the candidate compound can be used for treating or preventing Alzheimer's disease.

27. A method of screening for a pro-apoptotic compound comprising administering a candidate agent to a sample comprising a cell having a nucleic acid encoding Appoptosin and monitoring the sample for changes in the levels of Appoptosin, wherein a detectable change in the levels of Appoptosin is an indication that the candidate compound can be used for treating or preventing cancer.

28. A method of making an agent that modulates Appoptosin, comprising administering a candidate agent to a sample comprising cell having a nucleic acid encoding Appoptosin, detecting a change in the levels of Appoptosin, and making the candidate agent.

29. A method of identifying a subject having a leukemia or at risk of developing a leukemia, comprising detecting a level of Appoptosin in a blood or bone marrow cell of the subject that is at least 50% greater than a reference standard.

30. A method of monitoring the progress of a treatment for leukemia in a subject, comprising detecting the level of Appoptosin in a blood or bone marrow cell of the subject isolated before treatment and comparing it to the level of Appoptosin in a blood or bone marrow cell of the subject isolated after treatment, wherein a decrease in said Appoptosin levels after treatment is an indication of effective treatment.
